(19) United States
(12) Patent Application Publication
(10) Pub. No.: US 2026/0286370 A1
(43) Pub. Date: Sep. 24, 2026

(54) METHODS FOR RAPID AGROBACTERIUM-MEDIATED SUNFLOWER STABLE TRANSFORMATION

(71) Applicant: SYNGENTA CROP PROTECTION AG, Basel (CH)

(72) Inventors: Jianping Xu, Beijing (CN); Dawei Liang, Beijing (CN); Qin Wen, Beijing (CN)

(73) Assignee: SYNGENTA CROP PROTECTION AG, Basel (CH)

(21) Appl. No.: 19/473,966

(22) PCT Filed: Apr. 5, 2024
(86) PCT No.: PCT/US2024/023242

§ 371 Date: Oct. 9, 2025

(30) Foreign Application Priority Data
Apr. 14, 2023 (WO) PCT/CN2023/088375

(51) Int. Cl. C12N 15/82; A01H 4/00

(52) U.S. Cl. CPC C12N 15/8205; A01H 4/008

(57) ABSTRACT

This disclosure provides methods of genetic transformation of sunflower, which are based on Agrobacterium-mediated gene delivery to meristem cells of freshly imbibed/germinated sunflower seeds. One embodiment of the disclosure is methods of directly transforming Helianthus annuus with a heterologous polynucleotide from a mature seed comprising: generating an explant from a germinated a H. annuus seed to remove cotyledon and primary leaves with complete meristem and shoot radical; contacting the H. annuus explant comprising the cotyledon with an Agrobacterium comprising a heterologous polynucleotide in an infection medium supplemented with a plant hormone using a vacuum and sonication; co-culturing the explant from a germinated a H. annuus plant in the presence of the Agrobacterium in a medium lacking the plant hormone; culturing the mature seed explant in a regeneration medium comprising a selection agent; and culturing the seed with regenerated shoot with secondary roots in a medium lacking a selection agent.

### RELATED APPLICATION INFORMATION

This application claims priority to application PCT/CN2023/088375 filed Apr. 14, 2023, the contents of which are incorporated herein by reference in its entirety.

### TECHNICAL FIELD

The invention relates to the field of plant genetic engineering, in particular rapid Agrobacterium-mediated transformation of sunflowers.

### BACKGROUND

Sunflower (Helianthus annuus L.) is one of the major edible oilseed crops cultivated across the world. It is the fourth position in the total production of vegetable oils around the globe. However, crop/trait improvement of sunflower is much slower than other major crops (e.g., corn, rice, wheat, and soy) because of lacking stable and efficient transformation systems. To date, genetic improvement is via traditional breeding by leveraging native genetic diversity among sunflower varieties. This method is quite limited for trait improvement.

There are several public reports of sunflower transformation. Scltiammeijer et al. firstly generated transgenic sunflower events by grafting of transformed shoots onto non-transgenic rootstocks to recover transgenic seedlings, but the efficiency was quite low (Scltiammeijer et al. (1990) Plant Cell Rep. 9:55-60). Mushke et al. described transformation of sunflower embryonic axis explants; however, the protocol has low transformation efficiency and requires a long timeline (~103 days) (Mushke et al. (2019) Molecular Biology Reports). Thus, though sunflower transformation has been demonstrated in 1990s, the transformation efficiency is rarely low, and the timeline is 3 months or more. Germplasm independent transformation is lacking.

Accordingly, what is needed is a stable and efficient sunflower transformation system that can produce high quality transgenic events to facilitate sunflower trait research and crop improvement.

### SUMMARY

This disclosure provides methods of genetic transformation of sunflower, which are based on Agrobacterium-mediated gene delivery to meristem cells of freshly imbibed/germinated sunflower seeds.

One embodiment of the disclosure is directed to methods of directly transforming a Helianthus annuus plant with a heterologous polynucleotide from a mature seed comprising: generating an explant from a germinated a Helianthus annuus plant seed to remove one cotyledon and primary leaves with complete meristem and shoot radical; gently making wound at apical meristem region with the tip sharp end of scalpel; contacting the Helianthus annuus explant comprising one of the cotyledons with an Agrobacterium tumefaciens comprising a heterologous polynucleotide in an infection medium supplemented with a plant hormone optionally using a vacuum and sonication; co-culturing the explant from a germinated a Helianthus annuus plant in the presence of the Agrobacterium tumefaciens in a medium; culturing the mature seed explant in a regeneration medium comprising a selection agent (e.g. an antibiotic such as spectinomycin and bensulfuron-methyl); culturing the seed with regenerated shoot with or without secondary roots in a medium lacking a selection agent (e.g. an antibiotic such as spectinomycin and bensulfuron-methyl); and transplanting the explants into soil under conditions allowing for further development.

In certain embodiments, the methods include germinating a Helianthus annuus plant seed for about a day before generating the explant. The methods also include modifying Agrobacterium tumefaciens to comprise the heterologous polynucleotide, such as e.g., a gene.

In certain embodiments, the contacting the Helianthus annuus explant comprising the cotyledon with an Agrobacterium tumefaciens comprising a heterologous polynucleotide in an infection medium supplemented with a plant hormone is carried out using a vacuum and sonication. In some embodiments, the vacuum and sonication enhance infection efficiency. While a variety of different plant hormones may be used, in certain embodiments, the plant hormone comprises TDZ and/or BAP, Zeatin Riboside. In some embodiments, about 3-20 mg/L of the plant hormone are used. In some embodiments, the method comprises contacting the Helianthus annuus in an infection medium containing the plant hormone for about 4 hours or less.

In other embodiments, the co-culturing comprises culturing with or without light. For example, in certain embodiments, the co-culturing includes culturing under low light. The method may include co-culturing for about 4-6 days.

In certain embodiments, the culturing the mature seed explant in a regeneration medium comprises insertion of the cotyledon/meristem region into a solid medium in the presence of a selection agent (e.g., an antibiotic such as spectinomycin and bensulfuron-methyl), covering the seed with soil matrix and growing the seeds. In some embodiments, culturing lasts for about 7-14 days. In alternate embodiments, each day of culturing comprises culturing 16 hours in the presence of light and 8 hours in the presence of dark. In other embodiments, the culturing lasts for about 7-14 days and includes culturing in dark for about the first 2-3 days.

In some embodiments, the seed is cultured having regenerated shoot with or without secondary roots in a medium lacking a selection agent (e.g., an antibiotic). The culture is for about 3-7 days.

In other embodiments, the explants are transplanted into soil under conditions allowing for further development. This comprises growing the explants for about 2-3 weeks without any selection (e.g., any selection agent such as an antibiotic).

The method can also include a further step such as characterization of the transgenic plant and/or determining the presence of the heterologous polynucleotide in the transgenic plant.

The disclosure also includes a plant or plant part produced by the any of these methods. In addition, the disclosure includes a progeny seed produced by crossing such a plant of with a second plant or by selfing the plant. In addition, the disclosure includes a derivative, or a commodity product produced or obtained from the plant or plant part.

### BRIEF DESCRIPTION OF THE DRAWINGS

The foregoing summary, as well as the following detailed description of the invention, will be better understood when read in conjunction with the appended figures. For the purpose of illustrating the invention, the figures demonstrate embodiments of the present invention. It should be understood, however, that the invention is not limited to the precise arrangements, examples, and instrumentalities shown.

FIG. 1 outlines general steps of current methods used for transformation.

FIG. 2B and FIG. 2A outline the general steps of the methods of the disclosure.

FIG. 3 shows a picture of an explant used for infection. The explant was generated by removing the primary leaves and making a wound across the apical region.

FIG. 4A and FIG. 4B show explants after co-culture with agrobacterium for four days.

FIG. 5A and FIG. 5B show green fluorescent protein (GFP)expression of explants after co-culture.

FIGS. 6A-5D show GFP expression of explants after regeneration for 9 days (SF69 A).

FIGS. 7A-7C show explants for transplantation.

FIG. 8A and FIG. 8B show plantlets twenty-one days after transplantation.

FIG. 9 shows a construct map of vector 24545.

FIG. 10 shows a construct map of vector 18891.

FIG. 11A-D show GFP detection in transgenic sunflower plants. FIG. 11A and FIG. 11B show leaves of positive events (successful transformation) in white and green fluorescence in sunflower plants transformed with vector 24544. FIG. 11C and FIG. 11B show negative plant leaves (i.e., no transformation) in white and green fluorescence in sunflower plants unsuccessfully treated with vector 24545.

FIG. 12A-D show cyan fluorescent protein (CFP) detection in transgenic sunflower plants. FIG. 12A and FIG. 12B shows leaves of positive events (successful transformation) in white and blue fluorescence in sunflower plants transformed with vector 18891. FIG. 12C and FIG. 12D show negative plant's leaves in white and blue fluorescence in sunflower plants unsuccessfully treated with vector 18891.

FIG. 13A-13C show that transgenic plants grow normally. FIG. 13A shows plants SUET210200A004A and SUET210200A005A. FIG. 13B shows plant SUET210200A004A. FIG. 13C shows plant SUET210200A005A.

FIG. 14A-14C show that transgenic plants grow normally when grown for 23 days. FIG. 14A shows plant SUET210201A008A after 23 days of growth. FIG. 14B shows plant SUET210200A004A after 23 days of growth. FIG. 14C shows plant SUET210200A005A after 23 days of growth.

FIG. 15A-C show transgenic plants growing in green house for one month. FIG. 15A shows plant SUET210201A008A after one month of growth. FIG. 15B shows plant SUET210200A004A after one month of growth. FIG. 15C shows plant SUET210200A005A after one month of growth.

FIG. 16A-C show transgenic plants growing in green house for two months. FIG. 16A shows plant SUET210201A008A after two months of growth. FIG. 16B shows plant SUET210200A004A after two months of growth. FIG. 16C shows plant SUET210200A005A after two months of growth.

FIG. 17A-C show a comparison of transgenic plant seeds and wild-type plant seed. FIG. 17A shows transgenic seeds from plant SUET210201A008A on the left and wild-type seeds on the right. FIG. 17B shows transgenic seeds from plant SUET210200A004A on the left and wild-type seeds on the right. FIG. 17C shows transgenic seeds from plant SUET210200A005A, on the left and wild-type seeds on the right.

FIG. 18A and FIG. 18B show green fluorescent protein (GFP) expression in transgenic sunflower seeds. FIG. 18A shows a transgenic seed on the left and a wild type seed on the right. FIG. 18B shows GFP expression visible only in the transgenic seed and not the wild type seed.

FIG. 19A and FIG. 19B show CFP expression in transgenic sunflower seeds. FIG. 19A shows a transgenic seed on the left and a wild type seed on the right. FIG. 19B shows CFP expression visible only in the transgenic seed and not the wild type seed.

FIG. 20A and FIG. 20B show transgenic plants from SF81 grown for 21 days after transplantation.

FIG. 21A and FIG. 21B show transgenic plant grown in a pot. FIG. 21A shows a transgenic plant generated from one of the successfully transformed explants in SF-66-SP-24545-B (see Table 15). FIG. 21B shows a top view of the same plant.

### DETAILED DESCRIPTION

This disclosure provides new methods for generating stable sunflower transformations by using explant from imbibed seeds. These methods provide several advantages compared to current methods of transforming sunflower.

The disclosed methods provide a shortened turnaround process to generate transformed sunflower plants. In certain embodiments, the methods involve the following steps:
(a) Explant is generated from seeds germinated for about a day or two days by removing one cotyledon and primary leaves (with intact meristem and shoot radical);
(b) Infection of the explant is carried out by adding plant hormone such as thidiazuron (TDZ) and/or BAP, Zeatin Riboside to infection medium containing Agrobacterium and optionally vacuum and sonication are used to enhance infection efficiency;
(c) Co-culture for 4-6 days with or without light;
(d) Recover with selection agent (e.g., an antibiotic such as spectinomycin and bensulfuron-methyl) for 9-14 days, culture in dark for the first 2-3 days; and
(e) Transplant explants into soil after recovery for further development for 2-3 weeks.

Transgenic plants generated using the methods of the disclosure have normal development and agronomic performance (without early flowering or rooting issue). Furthermore, the transformation methods of the disclosure are shorter (taking less than 8 weeks to obtain transgenic plants) when compared with conventional methods.

### Definitions

Unless defined otherwise, all technical and scientific terms used herein have the same meaning as commonly understood by one of ordinary skill in the art to which the presently disclosed subject matter belongs.

Although the following terms are believed to be well understood by one of ordinary skill in the art, the following definitions are set forth to facilitate understanding of the presently disclosed subject matter.

Unless explicitly stated otherwise in the context, the singular forms “a,” “an,” and “the” as used herein include multiple references. Thus, for example, references to “a cell” include a plurality of such cells, and references to “the protein” include references to one or more proteins and their equivalents known to those skilled in the art, and so on. Unless explicitly stated otherwise, all technical and scientific terms used herein have the same meanings generally understood by those of ordinary skill in the art to which the present invention belongs.

As used in the specification and claims, the term “comprise” and grammatical variations thereof may include aspects of “consist of” and “substantially consist of”. “Comprise” and grammatical variations thereof may also mean “comprise, but not limited to.”

As used herein, the word “or” refers to any member of a particular list and also comprises any combination of members of the list.

As used herein, the term “and/or” refers to and encompasses any and all possible combinations of one or more of the associated listed items, as well as the lack of combinations when interpreted in the alternative (“or”).

A range may be expressed in the present invention as being from “about” one specific value and/or to “about” another specific value. When expressing ranges, other aspects include from one specific value and/or to other specific values. Similarly, when the value is expressed as an approximation, it should be understood that by using the antecedent “about,” the specific value forms another aspect. It should also be understood that the endpoints of each of the ranges are both significantly related to and independent of the other endpoint. It should also be understood that there are multiple values disclosed in the present invention and, in addition to the value itself, each value is also disclosed herein in the form of “about” the specific value. For example, if the value “10” is disclosed, “about 10” is also disclosed. It should also be understood that each unit between two specific units is also disclosed. For example, if 10 and 15 are disclosed, 11, 12, 13, and 14 are also disclosed.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). With regard to a temperature the term “about” means±1° C., preferably ±0.5° C. Where the term “about” is used in the context of this invention (e.g., in combinations with temperature or molecular weight values) the exact value (i.e., without “about”) is preferred.

The term “consists essentially of” (and grammatical variants thereof), as applied to a polynucleotide sequence of this invention, means a polynucleotide sequence that consists of both the recited sequence (e.g., SEQ ID NO) and a total of ten or less (e.g., 1, 2, 3, 4, 5, 6, 7, 8, 9, or 10) additional nucleotides on the 5′ and/or 3′ ends of the recited sequence such that the function of the polynucleotide is not materially altered. The total of ten or less additional nucleotides includes the total number of additional nucleotides on both ends added together. The term “materially altered,” as applied to polynucleotides of the invention, refers to an increase or decrease in ability to express the polynucleotide sequence of at least about 50% or more as compared to the expression level of a polynucleotide sequence consisting of the recited sequence.

As used herein, the term “apical dominance” refers to a phenomenon by which a main shoot dominates and inhibits the growth of axillary meristems. Apical dominance is thought to be caused by auxin, which moves downward toward the axillary meristems and inhibits their growth.

As used herein, the term “axillary bud” means an embryonic or organogenic bud located in the axil of a cotyledon or leaf. The axillary bud contains axillary meristem which is capable of developing into a branch shoot or flower clusters.

As used herein, the term “axillary meristem” refers to a region of a plant containing stem cells that is located on the lateral side of a stem of a plant, but is not located at the apex of the stem.

“Explant,” as used herein, refers to tissue, a piece of tissue, or pieces of tissue derived from a plant or a plant part, such as a seed. An explant can be a part of a plant, such as immature embryos, leaves meristems, or can be derived from a portion of the shoot, leaves, immature embryos or any other tissue of a plant or seed.

The term “introduced” as used herein, in connection to a plant, means accomplished by any manner including but not limited to; introgression, transgenic, Clustered Regularly Interspaced Short Palindromic Repeats modification (CRISPR), Transcription activator-like effector nucleases (TALENs) (Feng et al. 2013, Joung & Sander 2013), meganucleases, or zinc finger nucleases (ZFNs).

In the present invention, “nucleic acid” refers to a deoxyribonucleotide or ribonucleotide polymer in single-stranded or double-stranded form and, unless otherwise limited, encompasses known analogues (e.g., peptide nucleic acids) that have the basic properties of natural nucleotides in the following aspects: it hybridizes to single-stranded nucleic acids in a manner similar to that of naturally occurring nucleotides.

The term “a variant” and grammatical variations thereof refer to a substantially similar sequence. For nucleic acid molecules, variants comprise deletion and/or addition of one or more nucleotides at one or more sites in the native nucleic acid molecule, and/or substitution of one or more nucleotides at one or more sites in the native nucleic acid molecule.

The term “protein” refers to a polymer of amino acid residues. The term applies to amino acid polymers in which one or more amino acid residues are artificial chemical analogues of corresponding natural amino acids, and to natural amino acid polymers.

As used herein, a “native” nucleic acid molecule or protein comprises a naturally occurring nucleotide sequence or an amino acid sequence, respectively.

As used herein, the term “coding” or “encoding” and grammatical variations thereof are used to mean that a nucleic acid comprises the desired information, which is specified by the use of codons to direct the translation of nucleotide sequences (for example, leguminous sequences) into specific proteins. A nucleic acid coding a protein may comprise an untranslated sequence (e.g., an intron) within the translation region of the nucleic acid or may lack such an intermediate untranslated sequence (e.g., as in cDNA).

As used herein, the term “allele” refers to one of two or more different nucleotides or nucleotide sequences that occur at a specific locus.

A marker is “associated with” a trait when it is linked to it and when the presence of the marker is an indicator of whether and/or to what extent the desired trait or trait form will occur in a plant/germplasm comprising the marker. Similarly, a marker is “associated with” an allele when it is linked to it and when the presence of the marker is an indicator of whether the allele is present in a plant/germplasm comprising the marker. For example, “a marker associated with enhanced pathogen resistance” refers to a marker whose presence or absence can be used to predict whether and/or to what extent a plant will display a pathogen resistant phenotype.

As used herein, the terms “backcross” and “backcrossing” refer to the process whereby a progeny plant is repeatedly crossed back to one of its parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA,” pp. 41-53 (1994). The initial cross gives rise to the F1 generation. The term “BC1” refers to the second use of the recurrent parent, “BC2” refers to the third use of the recurrent parent, and so on.

A centimorgan (“cM”) is a unit of measure of recombination frequency. One cM is equal to a 1% chance that a marker at one genetic locus will be separated from a marker at a second locus due to crossing over in a single generation.

As used herein, the term “chromosomal interval defined by and including,” used in reference to particular loci and/or alleles, refers to a chromosomal interval delimited by and encompassing the stated loci/alleles.

As used herein, the terms “cross” or “crossed” refer to the fusion of gametes via pollination to produce progeny (e.g., cells, seeds, or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, the terms “cultivar” and “variety” refer to a group of similar plants that by structural or genetic features and/or performance can be distinguished from other varieties within the same species.

As used herein, the terms “desired allele,” “favorable allele” and “allele of interest” are used interchangeably to refer to an allele associated with a desired trait (e.g., ASR resistance).

As used herein, the terms “inhibit,” “reduce,” etc., and grammatical variations thereof refer to any reduction in the expression or function of a target gene product, including any relative reduction in the expression or function up to and including complete elimination of the expression or function of the target gene product.

The term “enhance” and grammatical variations thereof refer to improvement, increase, amplification, reproduction, rise and/or elevation to reduce one or more disease symptoms.

As used herein, the terms “increase,” “enhance” etc., and grammatical variations thereof are used to refer to any promotion or gain or increase in the expression, function, or activity of a product of a target gene (for example, a resistance gene) as compared to a susceptible plant, thereby providing increased resistance to one or more pathogens (for example, Phakopsora) or diseases (for example, rust). Additionally, as used herein, the term “cause” or “increase” and grammatical variations thereof may refer to a higher expression of a target gene product such that the level is increased by 10% or more, 50% or more, or 100%, relative to a cell or plant lacking the target gene or protein disclosed herein.

The term “immunity” or “immune” is used in the present invention to refer to the absence of any macroscopically visible disease symptoms. The term “partial resistance” is used in the present invention to refer to the presence of macroscopically visible lesions without or with limited spore formation and/or a reduction in the scope or degree of any disease symptoms and/or a delay in the progression of any disease symptoms, and may, for example, manifest a reduction in the number of lesions or lesions with reduced spore formation. As used herein, the term “susceptibility” or the phrase “lack of resistance” in terms of rust refers to the occurrence of a lesion in the case where the spore formation level is equal to or higher than the spore formation level observed in a reference standard, such as, for example, the variety Williams or Peking.

The term “resistance” is used herein to refer to the absence or reduction of one or more disease symptoms caused by plant pathogens in plants. Resistance may mean that disease symptoms, such as the number of diseased plants, defoliation, and associated yield loss, are reduced, minimized, or decreased when compared to plants susceptible to the diseases or plants that do not comprise effective resistance genes that reduce one or more disease symptoms. In addition, resistance may include prevention or delay of pathogen proliferation. Generally speaking, the term “resistance” includes immunity and partial resistance as defined above.

As used herein, the terms “enhanced pathogen resistance,” “enhanced plant pathogen resistance”, or “enhanced disease resistance” refers to an improvement, enhancement, or increase in a plant's ability to endure and/or thrive despite being infected with a disease (e.g., Asian soybean rust) as compared to one or more control plants (e.g., one or both of the parents, or a plant lacking a marker associated with enhanced pathogen resistance to respective pathogen/disease). Enhanced disease resistance includes any mechanism (other than whole-plant immunity or resistance) that reduces the expression of symptoms indicative of infection for a respective disease.

“A plant pathogen” and grammatical variations thereof can be used herein to refer to, for example, a fungal pathogen of the genus Phakopsora of the class Basidiomycetes (including Phakopsora pachyrhizi and Phakopsora meibomiae).

The term “disease resistance gene” or “resistance gene” is used in the present invention to refer to a gene encoding a protein capable of enhancing or improving the defense or immune system response in plants.

The term “orthologue” and grammatical variations thereof refer to genes derived from common ancestral genes and present in different species due to speciation.

The term “germplasm” is used in the present invention to refer to genetic material derived from an individual (e.g., a plant), a group of individuals (e.g., a plant germline, variety, or family), or a clone derived from a strain, variety, species, or culture. Germplasm can be part of an organism or a cell or can be isolated from an organism or a cell. Germplasm provides genetic material having a specific molecular composition that provides the physical basis for some or all of the genetic properties of an organism or cell culture

A “genetic map” is a description of genetic linkage relationships among loci on one or more chromosomes within a given species, generally depicted in a diagrammatic or tabular form. For each genetic map, distances between loci are measured by the recombination frequencies between them. Recombinations between loci can be detected using a variety of markers. A genetic map is a product of the mapping population, types of markers used, and the polymorphic potential of each marker between different populations. The order and genetic distances between loci can differ from one genetic map to another.

As used herein, the term “genotype” refers to the genetic constitution of an individual (or group of individuals) at one or more genetic loci, as contrasted with the observable and/or detectable and/or manifested trait (the phenotype). Genotype is defined by the allele(s) of one or more known loci that the individual has inherited from its parents. The term genotype can be used to refer to an individual's genetic constitution at a single locus, at multiple loci, or more generally, the term genotype can be used to refer to an individual's genetic make-up for all the genes in its genome. Genotypes can be indirectly characterized, e.g., using markers and/or directly characterized by nucleic acid sequencing.

As used herein, the term “germplasm” refers to genetic material of or from an individual (e.g., a plant), a group of individuals (e.g., a plant line, variety, or family), or a clone derived from a line, variety, species, or culture. The germplasm can be part of an organism or cell or can be separate from the organism or cell. In general, germplasm provides genetic material with a specific molecular makeup that provides a physical foundation for some or all of the hereditary qualities of an organism or cell culture. As used herein, germplasm may refer to seeds, cells (including protoplasts and calli) or tissues from which new plants may be grown, as well as plant parts that can be cultured into a whole plant (e.g., stems, buds, roots, leaves, etc.).

A “haplotype” is the genotype of an individual at a plurality of genetic loci, i.e., a combination of alleles. Typically, the genetic loci that define a haplotype are physically and genetically linked, i.e., on the same chromosome segment. The term “haplotype” can refer to polymorphisms at a particular locus, such as a single marker locus, or polymorphisms at multiple loci along a chromosomal segment.

As used herein, the term “heterozygous” refers to a genetic status wherein different alleles reside at corresponding loci on homologous chromosomes.

As used herein, the term “homozygous” refers to a genetic status wherein identical alleles reside at corresponding loci on homologous chromosomes.

As used herein, the term “hybrid” refers to a seed and/or plant produced when at least two genetically dissimilar parents are crossed.

As used herein, the term “inbred” refers to a substantially homozygous plant or variety. The term may refer to a plant or variety that is substantially homozygous throughout the entire genome or that is substantially homozygous with respect to a portion of the genome that is of particular interest.

As used herein, the term “indel” refers to an insertion or deletion in a pair of nucleotide sequences, wherein a first sequence may be referred to as having an insertion relative to a second sequence or the second sequence may be referred to as having a deletion relative to the first sequence.

As used herein, the terms “introgression,” “introgressing” and “introgressed” refer to both the natural and artificial transmission of a desired allele or combination of desired alleles of a genetic locus or genetic loci from one genetic background to another. For example, a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele may be a selected allele of a marker, a QTL, a transgene, or the like. Offspring comprising the desired allele can be repeatedly backcrossed to a line having a desired genetic background and selected for the desired allele, with the result being that the desired allele becomes fixed in the desired genetic background. For example, a marker associated with enhanced ASR tolerance may be introgressed from a donor into a recurrent parent that is not disease resistant. The resulting offspring could then be repeatedly backcrossed and selected until the progeny possess the ASR tolerance allele(s) in the recurrent parent background.

As used herein, the term “expression cassette” refers to a nucleotide capable of directing expression of a particular nucleic acid sequence in a host cell. In some embodiments, the expression cassette comprises, consists essentially of, or consists of one or more promoter sequences (e.g., one or more constitutive/inducible promoter sequences, one or more tissue- and/or organ-specific promoter sequences and/or one or more developmental stage-specific promoter sequences) operably linked to a nucleic acid of interest, which is operably linked to a termination sequence. Expression cassettes often comprise sequences required for proper translation of the nucleic acid sequence of interest in the host cell. The expression cassette may be chimeric in that at least one of its components is heterologous with respect to at least one of its other components. The expression cassette may be one that is naturally occurring but that has been obtained in a recombinant form useful for heterologous expression. Typically, however, the expression cassette is heterologous with respect to the host (i.e., the particular nucleic acid sequence of the expression cassette does not occur naturally in the host cell and must have been introduced into the host cell or an ancestor of the host cell by a transformation event).

As used herein, the term “genome editing agent” refers to an agent that is capable of inducing a deletion, insertion, indel, or other modification in the genome of a cell, e.g., by creating a single or double-stranded break in the genome. Examples of genome editing agents include CRISPR/Cas agents (e.g., Cas proteins and guide RNAs), transcription activator-like effector nucleases (TALENs), DNA-guided nucleases, meganucleases, recombinases, and zinc finger nucleases. Cas proteins include Cas9, Cas12a (also known as Cpf1), C2c1, C2c2, and C2c3, and functional variants thereof. Example Cas9 and Cas12a proteins include Streptococcus pyogenes Cas9 (SpCas9), Streptococcus thermophilus Cas9 (StCas9), Streptococcus pasteurianus (SpaCas9), Campylobacter jejuni Cas9 (CjCas9), Staphylococcus aureus (SaCas9), Francisella novicida Cas9 (FnCas9), Neisseria cinerea Cas9 (NcCas9), Neisseria meningitis Cas9 (NmCas9), Francisella novicida Cpf1 (FnCpf1), Acidaminococcus sp. Cpf1 (AsCpf1), or Lachnospiraceae bacterium ND2006 Cpf1 (LbCpf1). A “variant” of a Cas protein refers to a protein or polypeptide derivative of a Cas protein, e.g., a protein having one or more point mutations, insertions, deletions, truncations, a fusion protein, or a combination thereof. In certain embodiments, the Cas variant is a functional variant which substantially retains the nuclease activity of or has better nuclease activity than the wild-type Cas protein. Example guide RNAs include single guide RNAs and dual guide RNAs.

As used herein, the term “heterologous” refers to a polynucleotide/polypeptide at least a part of which originates from a foreign species, or, if from the same species, is substantially modified from its native form in composition and/or genomic locus by deliberate human intervention. Thus, a nucleotide sequence derived from an organism or species different from that of the cell into which the nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g., present in a different copy number, and/or under the control of different regulatory sequences than that found in the native state of the nucleic acid molecule. A nucleic acid sequence can also be heterologous to other nucleic acid sequences with which it may be associated, for example in a nucleic acid construct, such as e.g., an expression vector. As one nonlimiting example, a promoter may be present in a nucleic acid construct in combination with one or more regulatory element and/or coding sequences that do not naturally occur in association with that particular promoter, i.e., they are heterologous to the promoter.

As used herein, the term “in planta” when referring to a process or method step refers to a process or method step that is performed on a plant and not on excised or in vitro cultivated plant tissues or organs. For clarity, a plant includes those that have been wounded or have had one or more tissues removed, e.g., a plant having wounded axillary meristems and/or removed SAMs.

As used herein, the term “in planta transformation” refers to a transformation method that is performed on a plant without any tissue culture steps performed on any excised tissues or organs. For clarity, tissue culture steps do not include growing the plant on or in growth media, hydroponics, media plates, etc.

The terms “nucleic acid” or “polynucleotide” are used interchangeably herein and refer to any physical string of monomer units that can be corresponded to a string of nucleotides, including a polymer of nucleotides (e.g., a typical DNA polymer or polydeoxyribonucleotide or RNA polymer or polyribonucleotide), modified oligonucleotides (e.g., oligonucleotides comprising bases that are not typical to biological RNA or DNA, such as 2′-0-methylated oligonucleotides), and the like. In some embodiments, a nucleic acid or polynucleotide can be single-stranded, double-stranded, multi-stranded, or combinations thereof. Unless otherwise indicated, a particular nucleic acid or polynucleotide of the present invention optionally comprises or encodes complementary polynucleotides, in addition to any polynucleotide explicitly indicated. The nucleic acid can be present in a vector, such as in a cell, vims, or plasmid.

As used herein, the phrases “operably linked,” “operatively linked,” “operatively associated” or “in operative association” and the like, mean that elements of a nucleic acid construct such as an expression cassette or nucleic acid molecule are configured so as to perform their usual function. Thus, regulatory or control sequences (e.g., promoters) operatively associated with a nucleotide sequence are capable of effecting expression of the nucleotide sequence. For example, a promoter is operably linked with a coding sequence or functional RNA when it is capable of affecting the expression of that coding sequence or functional RNA (i.e., the coding sequence or functional RNA is under the transcriptional control of the promoter). Coding sequences in sense or antisense orientation can be operably-linked to regulatory sequences. The control sequences need not be contiguous with the nucleotide sequence of interest, as long as they function to direct the expression thereof.

Thus, for example, intervening untranslated, yet transcribed, sequences can be present between a promoter and a coding sequence, and the promoter sequence can still be considered “operably linked” to the coding sequence.

The term “plant” refers to any plant, particularly to agronomically useful plants (e.g., seed plants), and “plant cell” is a structural and physiological unit of the plant, which comprises a cell wall but may also refer to a protoplast. The plant cell may be in form of an isolated single cell or a cultured cell, or as a part of higher organized units such as for example, a plant tissue, or a plant organ differentiated into a structure that is present at any stage of a plant's development. A plant may be a monocotyledonous or dicotyledonous plant species.

The term “plant part” indicates a part of a plant, including single cells and cell tissues such as plant cells that are intact in plants, cell clumps and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as pollen, ovules, leaves, embryos, roots, root tips, anthers, flowers, fruits, stems, shoots, scions, rootstocks, seeds, protoplasts, calli, and the like. The term “plant part” also includes explants.

The term “progeny” refers to the descendant(s) of a particular cross. Typically, progeny result from breeding of two individuals, although some species (particularly some plants and hermaphroditic animals) can be selfed (i.e., the same plant acts as the donor of both male and female gametes). The descendant(s) can be, for example, of the F1, the F2, or any subsequent generation.

“Promoter” refers to a nucleotide sequence, usually upstream (5′) to its coding sequence, which controls the expression of the coding sequence by providing the recognition for RNA polymerase and other factors required for proper transcription. “Promoter regulatory sequences” consist of proximal and more distal upstream elements. Promoter regulatory sequences influence the transcription, RNA processing or stability, or translation of the associated coding sequence. Regulatory sequences include enhancers, promoters, untranslated leader sequences, introns, and polyadenylation signal sequences. They include natural and synthetic sequences as well as sequences that may be a combination of synthetic and natural sequences. An “enhancer” is a DNA sequence that can stimulate promoter activity and may be an innate element of the promoter or a heterologous element inserted to enhance the level or tissue specificity of a promoter. It is capable of operating in both orientations (normal or flipped) and is capable of functioning even when moved either upstream or downstream from the promoter. The meaning of the term “promoter” includes “promoter regulatory sequences.”

As used herein, the term “shoot apical meristem,” “shoot apex meristem” or “SAM” refers to a region of a plant containing stem cells that is located at the apex of a stem of a plant.

By “stably introducing” or “stably introduced” in the context of a polynucleotide introduced into a cell is intended the introduced polynucleotide is stably incorporated into the genome of the cell, and thus the cell is stably transformed with the polynucleotide.

“Stable transformation” or “stably transformed” as used herein means that a nucleic acid is introduced into a cell and integrates into the genome of the cell. As such, the integrated nucleic acid is capable of being inherited by the progeny thereof, more particularly, by the progeny of multiple successive generations. “Genome” as used herein also includes the nuclear, mitochondrial and the plastid genome, and therefore includes integration of the nucleic acid into, for example, the chloroplast genome. Stable transformation as used herein can also refer to a transgene that is maintained extrachromasomally, for example, as a minichromosome.

“Selection agent” refers to an agent (e.g., a chemical) that interacts with a selectable marker to give a plant cell a selective advantage. Example selection agents are known in the art and described herein, such as glyphosate, glufosinate, spectinomycin, bensulfuron-methyl, and kanamycin.

A “selectable marker” or “selectable marker gene” refers to a gene whose expression in a plant cell gives the cell a selective advantage. “Positive selection” refers to a transformed cell acquiring the ability to metabolize a substrate that it previously could not use or could not use efficiently, typically by being transformed with and expressing a positive selectable marker gene. This transformed cell thereby grows out of the mass of nontransformed tissue. Positive selection can be of many types from inactive forms of plant growth regulators that are then converted to active forms by the transferred enzyme to alternative carbohydrate sources that are not utilized efficiently by the nontransformed cells, for example mannose, which then become available upon transformation with an enzyme, for example phosphomannose isomerase, that allows them to be metabolized. Nontransformed cells either grow slowly in comparison to transformed cells or not at all. Other types of selection may be due to the cells transformed with the selectable marker gene gaining the ability to grow in presence of a negative selection agent, such as an antibiotic or an herbicide, compared to the ability to grow of non-transformed cells. A selective advantage possessed by a transformed cell may also be due to the loss of a previously possessed gene in what is called “negative selection.” In this, a compound is added that is toxic only to cells that did not lose a specific gene (a negative selectable marker gene) present in the parent cell (typically a transgene).

The term “transformation” as used herein refers to the transfer of a nucleic acid into a host cell, which includes integration into a chromosome, heritable extrachromosomal events, and transient transfer. In some particular embodiments, the introduction into a plant, plant part and/or plant cell is via bacterial-mediated transformation. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell Mol Biol Lett 7:849-858 (2002)).

As used herein, the term “transgenic” refers to any plant, plant cell, callus, plant tissue, or plant part that contains all or part of at least one heterologous polynucleotide. In some embodiments, all or part of the heterologous polynucleotide is stably integrated into a chromosome or stable extra-chromosomal element, so that it is passed on to successive generations.

Various publications, articles and patents are cited or described in the background and throughout the specification; each of these references is herein incorporated by reference in its entirety. All patents, published patent applications, and publications cited herein are incorporated by reference as if set forth fully herein.

Unless defined otherwise, all technical and scientific terms used herein have the same meaning commonly understood to one of ordinary skill in the art to which this invention pertains. Otherwise, certain terms used herein have the meanings as set forth in the specification.

### Methods of Transforming Sunflower

The disclosure provides methods of transforming sunflower seeds using Agrobacterium. The methods of the disclosure provide an efficient transformation system of sunflower starting from imbibed seeds. This system provides a short timeline with only 7 weeks or earlier resulting in transgenic plants that are normal and healthy without issue of early flowering and rooting. The methods are also germplasm independent.

The methods of the disclosure can be divided into a number of different stages. Each of these stages can include more than one method step. In certain embodiments, more than one method step can be encompassed by these stages. In other embodiments a single method step can encompass more than one stage.

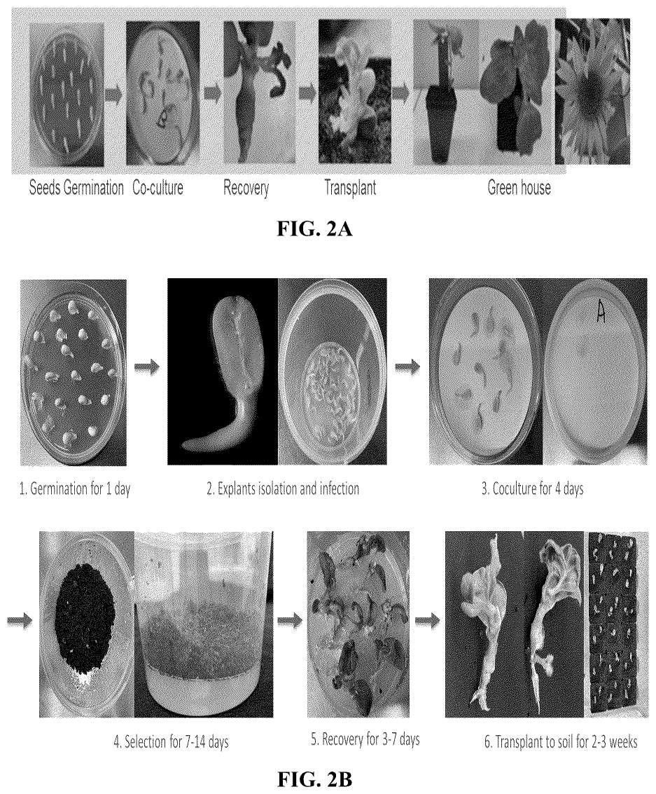

As illustrated in FIG. 2A, in certain embodiments of the disclosure the methods include seed germination, co-culture with Agrobacterium containing a nucleic acid of interest, recovery of the transfected sunflower seeds, transplantation of sunflower seeds transfected with the nucleic acid of interest, and optionally growth of plants from the seed.

In another embodiment of the disclosure, the methods include: (1) germination of mature sunflower seeds; (2) explant isolation and infection with Agrobacterium containing a nucleic acid of interest (e.g., a heterologous polynucleotide); (3) co-culturing (contacting) in the presence of the Agrobacterium containing a nucleic acid of interest (e.g., a heterologous polynucleotide); (4) selection for transfected sunflower seeds containing a nucleic acid of interest; (5) recovery of the transfected sunflower seeds; and (6) transplantation of the soil. In certain embodiments, the germination step lasts for about one day. In other embodiments, the co-culturing lasts for about four days. In further embodiments, the selection step lasts for 7-14 days. In additional embodiments, the recovery step lasts about 3-7 days. In yet further embodiments, the transplantation step lasts 2-3 weeks.

One embodiment of the disclosure is directed to methods of directly transforming a Helianthus annuus plant with a heterologous polynucleotide from a mature seed including at least the steps of: generating an explant from a germinated a Helianthus annuus plant seed to remove cotyledon and primary leaves with complete meristem and shoot radical; gently making wound at apical meristem region with the tip sharp end of scalpel; contacting the Helianthus annuus explant comprising the cotyledon with an Agrobacterium tumefaciens comprising a heterologous polynucleotide in an infection medium supplemented with a plant hormone optionally using a vacuum and sonication; co-culturing the explant from a germinated a Helianthus annuus plant in the presence of the Agrobacterium tumefaciens in a medium lacking the plant hormone; culturing the mature seed explant in a regeneration medium comprising a selection agent (e.g. an antibiotic such as spectinomycin and bensulfuron-methyl); culturing the seed with regenerated shoot with or without secondary roots in a medium lacking a selection agent (e.g. an antibiotic such as spectinomycin and); and transplanting the explants into soil under conditions allowing for further development.

### Explant Generation and Germination

The methods of the disclosure involve generating an explant from a germinated Helianthus annuus plant seed to remove one cotyledon and primary leaves with complete meristem and shoot radical.

In certain embodiments, the methods include germinating a Helianthus annuus plant seed for about a day prior to generating the explants.

In some embodiments, the generating of the explant involves obtaining and sterilizing sunflower seeds without husks. The sterilizing may be carried out via conventional sterilization methods, such as those described in the Examples below.

In certain embodiments, the generation of the explant involves removal of at least one cotyledon of the germinated Helianthus annuus seeds to expose the shoot apex with its two primary leaves. The two primary leaves are removed completely to expose the apical meristem and the apical region is gently wounded.

Infection with Agrobacterium

Once the explant is generated, for example, as described above, the explant is infected with an Agrobacterium, such as e.g., Agrobacterium tumefaciens which contains a heterologous polynucleotide. In some embodiments, the heterologous polynucleotide is a gene.

The methods also include modifying the Agrobacterium, such as e.g., Agrobacterium tumefaciens, to comprise the heterologous polynucleotide.

In one embodiment, the method comprises contacting the Helianthus annuus explant comprising the cotyledon with an Agrobacterium tumefaciens comprising a heterologous polynucleotide in an infection medium supplemented with a plant hormone without using a vacuum and sonication. In another embodiment, the method comprises contacting the Helianthus annuus explant comprising the cotyledon with an Agrobacterium tumefaciens comprising a heterologous polynucleotide in an infection medium supplemented with a plant hormone using a vacuum and sonication. In certain embodiments, the vacuum and sonication enhance infection efficiency.

The method also includes preparing the Agrobacterium for infection (i.e., prior to contacting). The step of preparing may include growing the Agrobacterium on a suitable culture medium, such as e.g., YP medium. In one embodiment, the preparing of the Agrobacterium includes culturing in a suitable culture medium, such as e.g., YP medium supplemented with an antibiotic, for about at three days prior to infection (contacting). One day before contacting the Agrobacterium are cultured in a new suitable culture medium. Prior to infection, the Agrobacterium are transferred into a liquid infection medium. In one embodiment, the infection medium contains MS basal salts and B5 vitamins, about 20 g/L of sucrose, about 10 g/L of glucose, about 4 g/L of MES, about 2 mg/L of zeatin riboside, about 3-10 mg/L of TDZ, about 80 mg/L of acetosyringone (AS), and about 150 mg/L of dithiothreitol (DTT) at a pH of about 5.5.

In some embodiments, the contacting the Helianthus annuus explant comprising the cotyledon with an Agrobacterium tumefaciens includes placing the explants into a liquid infection medium containing the Agrobacterium. The medium is supplemented with a plant hormone. Suitable plant hormones for use in the contacting step include thidiazuron (1-phenyl-3-(1,2,3-thiadiazol-5-yl)urea) or a derivative thereof or a cytokinin such as 6-benzylaminopurine (BAP) or a derivative thereof. The medium may be further supplemented with for example 10% F68.

In one embodiment, the infection medium is supplemented with TDZ and/or BAP. In certain embodiments, medium is supplemented with about 3-20 mg/ml, alternatively about 3-10 mg/ml, alternatively about 3-15 mg/ml of the plant hormone (e.g., TDZ and/or BAP).

The medium containing the Agrobacterium and the explant(s) are then placed under a vacuum and sonicated. In one embodiment, the medium containing the Agrobacterium and the explant(s) are placed under a vacuum at 800-900 mbar for a about 5-30 minutes, alternatively, about 5-25 minutes alternatively, about 20 minutes, and then sonicated at about 45 kHz with gently shaking.

In some embodiments, the contacting includes culturing under shaking after the placing under the vacuum and sonication. The culturing includes culturing for about 2-3 hour and the shaking includes about 50-80 rpm. In certain embodiments, contacting the Helianthus annuus in an infection medium containing the plant hormone lasts for about 4 hours or less.

In some embodiments of the methods, the contacting step with Agrobacterium comprises an infection step and an incubation step. In some embodiments of the method, the infection step is performed for at least 30 minutes, e.g., 30 minutes to 24 hours, such as 1-12, 2-12, 3-12, 4-12, 5-12, 6-12, 7-12, 8-12, 9-12, 10-12, 11-12, 1-11, 2-11, 3-11, 4-11, 5-11, 6-11,7-11, 8-11, 9-11, 10-11, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2 hours, and the incubation step is performed in darkness or in light or in a light/dark cycle for at least 1 day, at least 2 days, at least 3 days, at least 4 days, at least 5 days, at least 6 days, at least 7 days or more, e.g., 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2 days. In some embodiments, the infection step comprises contacting the wounded axillary meristem(s) with a solution, gel, absorbable material, or other material that contains the Agrobacterium. In some embodiments, the infection step occurs for 5-12 hours. In some embodiments, the incubation step is performed in darkness for 3-7 days. In some embodiments, after incubation, antibiotics (e.g., Timentin, Cefotaxime and/or Vancomycin) are applied to eliminate the Agrobacterium.

Agrobacterium-mediated transformation is a method for used transforming plants. Agrobacterium-mediated transformation typically involves transfer of a binary vector carrying the foreign DNA of interest to an appropriate Agrobacterium strain that may depend on the complement of vir genes carried by the host Agrobacterium strain either on a co-resident Ti plasmid or chromosomally (see, e.g., Uknes et al. 1993, Plant Cell 5:159-169). The transfer of the recombinant binary vector to Agrobacterium can be accomplished, e.g., by a tri-parental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (see, e.g., Hofgen and Willmitzer 1988, Nucleic Acids Res 16:9877).

### Co-Culturing

After the Helianthus annuus explant comprising the cotyledon is contacted with the Agrobacterium tumefaciens in a medium supplemented with a plant hormone, the resultant explant from a germinated a Helianthus annuus plant is culture in the presence of the Agrobacterium tumefaciens in a medium lacks a plant hormone.

In other words, the methods also include co-culturing the explant from a germinated a Helianthus annuus plant in the presence of the Agrobacterium tumefaciens after removal of the plant hormone.

In certain embodiments of the methods, the co-culturing lasts for about 4-6 days, alternatively about 4 days. In certain embodiments, during the co-culturing the culture conditions are varied. For example, in certain embodiments, the co-culturing comprises culturing with or without light. In some embodiments, the co-culturing includes culturing under low light. In other embodiments, the co-culturing lasts for about 3-6 days and involves growing the explants without light.

In some embodiments, the step of co-culturing also includes removal of the explants from the infection medium.

The co-culturing is carried out in a suitable co-cultivation medium. In some embodiments, the cultivation medium is SFCoC, which may be supplemented with MS basal salts and vitamins, MS iron, about 1 mg/L of zeatin, about 10 g/L of sucrose, about 5 g/L of glucose, about 2 g/L of MES 2 g/L, about 20 mg/L of acetosyringone, and about 0.5 mg/ml of silver nitrate.

### Regeneration

As illustrated in FIG. 2B and FIG. 2A after co-culture, the transformed Helianthus annuus shoot is regenerated. Regeneration includes culturing the mature seed explant in a regeneration medium comprising a selection agent followed culturing the seed with regenerated shoot with secondary roots in a medium lacking a selection agent.

In some embodiments, the culturing the mature seed explant in a regeneration medium comprises insertion of the cotyledon/meristem region into a solid medium in the presence of a selection agent, covering the seed with matrix and growing the seeds. In certain embodiments, the culturing of the mature seed explant comprises culturing for about 7-14 days, alternatively for about 8-10 days, alternately 7-11 days, alternatively 7-12 days. In alternate embodiments, the culturing of the mature seeds comprises culturing in dark for about the first 2-3 days. In other embodiments, each day of culturing the mature seed explant comprises culturing about 16 hours in the presence of light and about 8 hours in the presence of dark.

In certain embodiments, the culturing of the mature seed explants comprises culturing in a medium supplemented with a plant hormone such as e.g., BAP. For example, the medium comprises SFR1 medium (Gamborg B5 basal salts and B5 vitamins, MS Iron 200×, about 20 g/L of sucrose, about 1 g/L of MES, about 50 mg/L of timentin 1, about 150 mg/L of carbenicillin, about 7.5 g/L of agar, about of 50 mM lipoic acid, about 1-4 mg/L BAP, and about 50-800 mg/L of spectinomycin).

The methods also include culturing the seed with regenerated shoot with or without secondary roots in a medium lacking a selection agent (e.g., an antibiotic such as spectinomycin and bensulfuron-methyl) for a suitable period of time such as, e.g., culturing for about 3-7 days. In some embodiment, the medium lacking the selection agent is SRi medium.

### Transplanting

After regeneration (i.e., culturing the mature seed explant in a regeneration medium comprising a selection agent followed by culturing the seed with regenerated shoots in a medium lacking a selection agent), explants are transplanted into soil under conditions allowing for further development.

When the explants have shoot with roots, they are ready for transplanting to soil.

In some embodiments, the transplanting the explants into soil under conditions allowing for further development comprises growing the explants for about 2-3 weeks without any selection.

Suitable conditions allowing for further development include but are not limited to about 27° C. in daytime for about 20 hours and 20° C. at night for about 4 hours.

In some embodiments, transplanting includes spraying a selection agent. Accordingly in other embodiments, the transplanting the explants into soil under conditions allowing for further development comprises growing the explants with a selection agent. For example, the selection agent is sprayed each day after transplanting.

In one embodiment, the selection agent is an antibiotic. In other embodiments, the selection agent is spectinomycin and/or bensulfuron-methyl.

In certain embodiments, the methods include characterization of the transgenic plant and/or selection of the transgenic plant.

In some embodiments, plants, plant parts, and plant cells transformed with a heterologous polynucleotide using the methods of the disclosure can be selected, e.g., using selectable markers present in the heterologous polynucleotide. In some embodiments, the plants, plant parts, and plant cells transformed with a heterologous polynucleotide are selected using one or more selection steps or selection agents described in the Examples.

Examples of selectable markers include, but are not limited to, genes that provide resistance or tolerance to antibiotics such as kanamycin (Dekeyser et al. 1989, Plant Phys 90: 217-23), spectinomycin (Svab and Maliga 1993, Plant Mol Biol 14: 197-205), streptomycin (Maliga et al. 1988, Mol Gen Genet 214: 456-459), hygromycin B (Waldron et al. 1985, Plant Mol Biol 5: 103-108), bleomycin (Hille et al. 1986, Plant Mol Biol 7: 171-176), sulphonamides (Guerineau et al. 1990, Plant Mol Biol 15: 127-136), streptothricin (Jelenska et al. 2000, Plant Cell Rep 19: 298-303), or chloramphenicol (De Block et al. 1984, EMBO J 3: 1681-1689). Other selectable markers include genes that provide resistance or tolerance to herbicides, such as the S4 and/or Hra mutations of acetolactate synthase (ALS) that confer resistance to herbicides including sulfonylureas, imidazolinones, triazolopyrimidines, and pyrimidinyl thiobenzoates; 5-enol-pyrovyl-shikimate-3-phosphate-synthase (EPSPS) genes, including but not limited to those described in U.S. Pat. Nos. 4,940,935, 5,188,642, 5,633,435, 6,566,587, 7,674,598 (as well as all related applications) and the glyphosate N-acetyltransferase (GAT) which confers resistance to glyphosate (Castle et al. 2004, Science 304:1151-1154, and U.S. Pub. App. Nos. 2007/0004912, 2005/0246798, and 2005/0060767); BAR which confers resistance to glufosinate (see e.g., U.S. Pat. No. 5,561,236); aryloxy alkanoate dioxygenase or AAD-1, AAD-12, or AAD-13 which confer resistance to 2,4-D; genes such as Pseudomonas HPPD which confer HPPD resistance;

Sprotophorphyrinogen oxidase (PPO) mutants and variants, which confer resistance to peroxidizing herbicides including fomesafen, acifluorfen-sodium, oxyfluorfen, lactofen, fluthiacet-methyl, saflufenacil, flumioxazin, flumiclorac-pentyl, carfentrazone-ethyl, sulfentrazone); and genes conferring resistance to dicamba, such as dicamba monoxygenase (Herman et al. 2005, J Biol Chem 280: 24759-24767 and U.S. Pat. No. 7,812,224 and related applications and patents). Other examples of selectable markers can be found in Sundar and Sakthivel (2008, J Plant Physiology 165: 1698-1716), herein incorporated by reference. Additional selectable markers for use in the disclosure are known in the art such as Phosphinothricin N-acetyl transferase (PAT) and Aminoglycoside 3′-adenylyiltransferase (ad) (see, e.g., Rosellini (2012) Selectable Markers and Reporter Genes: A Well-Furnished Toolbox for Plant Science and Genetic Engineering, Critical Reviews in Plant Sciences, 31:5, 401-453).

Other selection systems include using drugs, metabolite analogs, metabolic intermediates, and enzymes for positive selection or conditional positive selection of transgenic plants. Examples include, but are not limited to, a gene encoding phosphomannose isomerase (PMI) where mannose is the selection agent, or a gene encoding xylose isomerase where D-xylose is the selection agent (Haldrup et al. 1998, Plant Mol Biol 37: 287-96). Finally, other selection systems may use hormone-free medium as the selection agent. One non-limiting example the maize homeobox gene kn1, whose ectopic expression results in a 3-fold increase in transformation efficiency (Luo et al. 2006, Plant Cell Rep 25: 403-409). Examples of various selectable markers and genes encoding them are disclosed in Miki and McHugh (J Biotechnol, 2004, 107: 193-232; incorporated by reference).

In some embodiments of the disclosure, the selectable marker may be plant derived. An example of a selectable marker which can be plant derived includes, but is not limited to, 5-enolpyruvylshikimate-3-phosphate synthase (EPSPS). The enzyme 5-enolpyruvylshikimate-3-phosphate synthase (EPSPS) catalyzes an essential step in the shikimate pathway common to aromatic amino acid biosynthesis in plants. The herbicide glyphosate inhibits EPSPS, thereby killing the plant. Transgenic glyphosate-tolerant plants can be created by the introduction of a modified EPSPS transgene which is not affected by glyphosate (for example, U.S. Pat. No. 6,040,497; incorporated by reference). Other examples of a modified plant EPSPS which can be used as a selectable marker in the presence of glyphosate includes a P106L mutant of rice EPSPS (Zhou et al. 2006, Plant Physiol 140: 184-195) and a P106S mutation in goosegrass EPSPS (Baerson et al. 2002, Plant Physiol 129: 1265-1275). Other sources of EPSPS which are not plant derived and can be used to confer glyphosate tolerance include but are not limited to an EPSPS P101S mutant from Salmonella typhimurium (Comai et al. 1985, Nature 317: 741-744) and a mutated version of CP4 EPSPS from Agrobacterium sp. Strain CP4 (Funke et al. 2006, PNAS 103: 13010-13015). Although the plant EPSPS gene is nuclear, the mature enzyme is localized in the chloroplast (Mousdale and Coggins 1985, Planta 163:241-249). EPSPS is synthesized as a preprotein containing a transit peptide, and the precursor is then transported into the chloroplast stroma and proteolytically processed to yield the mature enzyme (della-Cioppa et al. 1986, PNAS 83: 6873-6877). Therefore, to create a transgenic plant which has tolerance to glyphosate, a suitably mutated version of EPSPS which correctly translocates to the chloroplast could be introduced. Such a transgenic plant then has a native, genomic EPSPS gene as well as the mutated EPSPS transgene. Glyphosate could then be used as a selection agent during the transformation and regeneration process, whereby only those plants or plant tissue that are successfully transformed with the mutated EPSPS transgene survive.

In some embodiments, the selection agent is an antibiotic such as spectinomycin and bensulfuron-methyl.

In some embodiments of the method, the heterologous polynucleotide comprises a selectable marker and the method further comprises contacting the plant with a selection agent to eliminate or reduce untransformed tissue. In some embodiments, the selection agent is an herbicide, an antibiotic, or a non-metabolizable sugar. In some embodiments, the selection agent is glyphosate, glufosinate, spectinomycin, bensulfuron-methyl, imazapyr, D-xylose, mannose, or kanamycin. In some embodiments, the selectable marker is EPSPS, and the selection agent is glyphosate.

In some embodiments of the method, the contacting with the selection agent comprises adding the selection agent to a medium (e.g., soil or hydroponics) in which the plant is growing (e.g., by watering or applying to the soil or other medium a composition comprising the selection agent, such as between 1 μM to 1 M of a selection agent, e.g., 100 μM to 500 μM of glyphosate), spraying the plant with the selection agent (e.g., with a sprayable composition comprising the selection agent, such as 1 μM to 1 M of a selection agent, e.g., between 10 μM to 50 mM glyphosate), or applying the selection agent (such as between 1 μM to 1 M of a selection agent, e.g., 100 μM to 200 μM glyphosate or 10 μM to 100 μM Bensulfuron-methyl) to the regenerated axillary meristem (e.g., using a solution, gel, absorbable material (e.g., cotton ball) or other material that can release the selection agent (such as onto the wounded axillary meristem and/or regenerated axillary meristem). In some embodiments, the contacting with the selection agent occurs for at least one day, at least one week, at least two weeks, at least three weeks, at least four weeks, at least five weeks, or longer. In some embodiments, the contacting with the selection agent occurs for between 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, or 5-6 weeks. In some embodiments, the contacting with the selection agent occurs for between 1 day to 6 weeks. In some embodiments, the contacting with the selection agent occurs for between 3-6 weeks.

In other embodiments of the method, the method further comprises performing an assay on a sample of the regenerated axillary meristem to assess the presence or absence of transformed cells in the sample and/or to assess the number of transformed cells in the sample. Example assays include fluorescent protein detection, qPCR, real-time PCR, immunoassays, and the like.

In alternate embodiments of the method, the method further comprises growing the plant to produce a seed (e.g., one seed, two seeds, ten seeds, twenty seeds, fifty seeds or more) optionally comprising at least part of the heterologous polynucleotide and harvesting the seed. In some embodiments, all seeds produced by the plant comprise at least part of the heterologous polynucleotide. In further embodiments, at least one seed, or more seeds (e.g., at least 10%, at least 20%, at least 30%, at least 40%, at least 50%, at least 60%, at least 70%, at least 80%, or at least 90%) of the seeds, produced by the plant comprise at least part of the heterologous polynucleotide. In additional embodiments of the method, the method further comprises growing the seed(s) to produce a progeny plant(s), optionally comprising at least part of the heterologous polynucleotide.

In further embodiments of the method, the heterologous polynucleotide encodes a genome editing agent, e.g., a CRISPR/Cas's agent, a TALEN, a DNA-guided nuclease, a mega nuclease, a recombinase, or a zinc finger nuclease. In alternate embodiments of the method, the heterologous protein comprises a genome editing agent, e.g., a Cas protein, a TALEN, a DNA-guided nuclease, a meganuclease, a recombinase, or a zinc finger nuclease. In some embodiments, the heterologous polynucleotide comprises one or more polynucleotides encoding a Cas protein and/or a guide RNA. In additional embodiments, the heterologous polynucleotide comprises one or more guide RNAs, optionally wherein the heterologous polynucleotide is comprised within a ribonucleoprotein (RNP) with a Cas protein. In further embodiments, the Cas protein is Cas9 or Cas12a, or a functional variant thereof.

In some embodiments of the method, the heterologous polynucleotide comprises an expression cassette comprising a coding sequence. In other embodiments of the method, the coding sequence encodes a protein or non-coding RNA of interest. In alternate embodiments, the protein or non-coding RNA of interest confers one or more desired traits on a plant, such as enhanced growth, enhanced yield, drought tolerance, salt tolerance, herbicide tolerance, insect resistance, pest resistance, disease resistance, temperature tolerance, enhanced nitrogen utilization and the like. In some embodiments, the coding sequence encodes a genome editing agent, such as a Cas protein and/or a guide RNA. In some embodiments, the heterologous polynucleotide comprises a coding sequence encoding a protein or non-coding RNA of interest and a coding sequence a selection marker. In additional embodiments of the method, the expression cassette further comprises a promoter operably linked to the coding sequence(s). The promoter may be, e.g., a constitutive promoter, a tissue-specific promoter, or an inducible promoter.

Other aspects of the disclosure relate to a plant or plant part produced by any of the methods described above or elsewhere herein, including in the Examples. Other aspects of the disclosure relate to progeny seed produced by crossing the plant produced by any of the methods described above or elsewhere herein with a second plant or by selfing the plant. Other aspects of the disclosure relate to a derivative, or a commodity product produced or obtained from the plant or plant part produced by any of the methods described above or elsewhere herein. In some embodiments, the commodity product is selected from the group consisting of whole or processed seeds, flour, protein isolates, concentrates, liquids, syrups, pastes, sauces or other food or product produced from the plant or plant part.

### Examples

The following examples further describe the invention, but do not limit the invention. The test materials used in the following examples, unless otherwise specified, were purchased from general biochemical reagent stores. For the quantitative tests in the following examples, three repeated tests were set, and the results were averaged.

### Example 1: Conventional Sunflower Transformation Process

Pilot studies on sunflower transformation with conventional in vitro processes commonly used to transform plants were conducted. However, these studies were unable to generate high quality transgenic events because of abnormal development during tissue culture, usual early flowering, and no roots. Reported conventional sunflower transformation protocols have low transformation frequency (0.06% to 0.52%) and are often associated with chimeric expression of the introduced gene (Alibert et al. 1999; Muller et al. 2001; Lucas et al. 2000; Hewezi et al. 2001; Molinier et al. 2002; Bidney et al. 1992; Knittel et al. 1994; Malone-Schoneberg et al. 1994; Burrus et al. 1996). Rao and Rohini (1999) used embryo axis as explant to generate transgenic shoots and resulted in 2% transformation frequency, but the rooting efficiency was quite low.

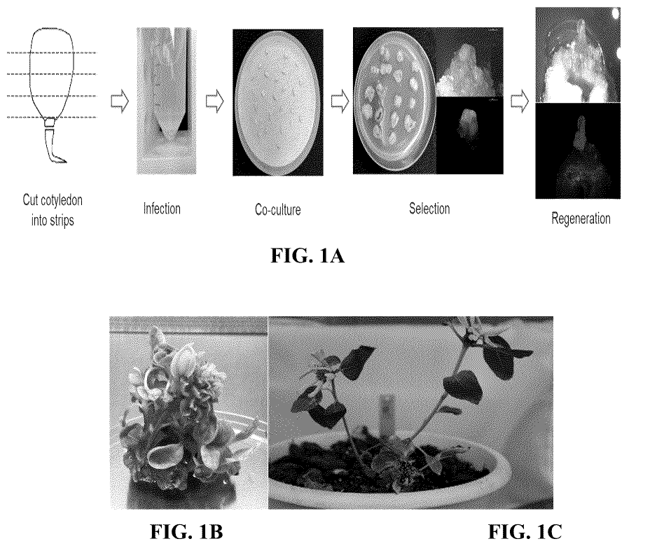

The most efficient regeneration and transformation system reported using cotyledon as explants was evaluated. After optimizing multiple transformation parameters (Table1, Table 2 and Table 3) it was possible to efficiently generate shoots (>80% shoot induction rate), however, most shoots were negative and only ~2% shoots were transgenic (with GFP signals), and the positive shoots did not survive because of low rooting and termination of growth during tissue culture (Table 4). FIG. 1A shows a pilot test of the conventional process.

TABLE 1
Shoot induction rate of different part of cotyledons

| Part of cotyledon | Adaxial | Abaxial | Cutting side |
|---|---|---|---|
| Shoot induction rate (%) | 42.8 | 9.4 | 42.2 |

TABLE 2
Shoot induction rate of different plant hormone

| | | BAP, | BAP, NAA, 24-epiBL, |
|---|---|---|---|
| Plant hormone | BAP | NAA | GA3 |
| Shoot induction rate (%) | 2 | 52.2 | 73.4 |

TABLE 3
Shoot induction rate of different explant stage

| | | | | |
|---|---|---|---|---|
| Germination time (h) | 2 (in water) | 24 | 30 | 48 |
| Shoot induction rate (%) | 82.2 | 58.1 | 35.4 | 11.1 |

TABLE 4
Summary of pilot test with conventional process

| | | | AS | | | | | | | | Pre- | Trans- |
|---|---|---|---|---|---|---|---|---|---|---|---|---|
| | | | (μM) | CoC | CoC | Inf | Inf L- | Inf | | Re- | treatment | genic |
| Treat- | Infection | | in | time | con- | DTT | Cystine | F68 | | covery | with SIM | shoots |
| ment | method | OD600 | CoC | (d) | dition | (mg/L) | (g/L) | (%) | Washing | time (d) | (d) | (%) |
| 1 | Vaccum 10 | 0.25 | 50 | 3 | light 16 h/ | 0 | 0 | 0 | Y | 0 | 0 | 0 |
| | min + | | | | dark 8 h | | | | | | | |
| | infection 60 | | | | | | | | | | | |
| | min | | | | | | | | | | | |
| 2 | Vaccum 10 | 0.25 | 50 | 3 | light 16 h/ | 150 | 0.4 | 0.02 | N | 4 | 1 | 0 |
| | min + | | | | dark 8 h | | | | | | | |
| | infection 60 | | | | | | | | | | | |
| | min | | | | | | | | | | | |
| 3 | Vaccum 10 | 0.25 | 150 | 4 | completely | 0 | 0 | 0 | N | 4 | 1 | 0 |
| | min + | | | | dark | | | | | | | |
| | infection 60 | | | | | | | | | | | |
| | min | | | | | | | | | | | |
| 4 | Vaccum 10 | 0.5 | 50 | 4 | completely | 0 | 0.4 | 0.02 | Y | 0 | 1 | 0 |
| | min + | | | | dark | | | | | | | |
| | infection 60 | | | | | | | | | | | |
| | min | | | | | | | | | | | |
| 5 | Vaccum 10 | 0.5 | 150 | 3 | completely | 150 | 0 | 0.02 | Y | 4 | 0 | 2.1 |
| | min + | | | | dark | | | | | | | |
| | infection 60 | | | | | | | | | | | |
| | min | | | | | | | | | | | |
| 6 | infection 60 | 0.5 | 150 | 4 | light 16 h/ | 150 | 0.4 | 0 | N | 0 | 0 | 0 |
| | min | | | | dark 8 h | | | | | | | |
| 7 | infection 60 | 0.25 | 150 | 4 | light 16 h/ | 0 | 0.4 | 0.02 | Y | 4 | 0 | 0 |
| | min | | | | dark 8 h | | | | | | | |
| 8 | infection 60 | 0.25 | 150 | 3 | completely | 150 | 0.4 | 0 | Y | 0 | 1 | 0 |
| | min | | | | dark | | | | | | | |
| 9 | infection 60 | 0.25 | 50 | 4 | completely | 150 | 0 | 0.02 | N | 0 | 0 | 0.9 |
| | min | | | | dark | | | | | | | |

Early conventional transformation experiments started from mature seeds. The mature seeds generated very low transformation efficiency and no plant was able to produce viable T1 transgenic seeds, as shown in FIGS. 1B (deformed plants without root) and 1C (precocious flowering plants).

TABLE 5
Transformation with conventional in vitro processes

| | | % of Explant | % of explant | % of Explant |
|---|---|---|---|---|
| | # of | Producing | producing | Producing |
| Genotype | Explants | green buds | shoots | Plant |
| B531 | 26 | 7.7 | 0.0 | 0.0 |
| F75400 | 58 | 17.2 | 0.0 | 0.0 |
| F703RM1 | 68 | 27.9 | 4.4 | 1.5 |
| FS5698B | 45 | 15.6 | 6.7 | 6.7 |
| BD40713MH | 43 | 7.0 | 0.0 | 0.0 |
| F73100 | 35 | 5.7 | 0.0 | 0.0 |
| F564 | 36 | 5.6 | 0.0 | 0.0 |
| BE78079 | 48 | 14.6 | 2.1 | 0.0 |

6.7% of TF was achieved with FS5698B, but had early flowering issue with no viable T1 seeds obtained

### Example 2: Generation of Transgenic Sunflowers by Agrobacterium Transformation (Protocol V1)

A variation of the sunflower transformation process was used to generate transgenic sunflowers expressing GFP and CFP. The transformation process used in this example is schematically outlined in FIG. 2A.

### (1) Seed Germination

First, the seeds were sterilized. Dry sunflower seeds without husk were soaked in 75% ethyl alcohol for 2 minutes. The seeds were transferred to a solution containing 3.5% Clorox with 0.04% silwet-77 for 15 min. Then, the seeds were rinsed with sterilized water at least 3 times.

The sterilized sunflower seeds were transferred to SF Germ medium (Gamborg B5 basal salts and vitamins, sucrose 20 g/L, agar 8 g/L, pH=5.6) with 20 seeds per petri dish and incubated at 25° C. in the dark for 20 hours.

### (2) Agrobacterium Preparation

Agrobacterium from storage at −80° C. were streaked to YP (Yeast Peptone) medium with 100 mg/L spectinomycin and 50 mg/L kanamycin at least 3 days before infection. They were re-streaked to a new YP medium plate with some concentrations of spectinomycin and kanamycin 1 day before infection. The Agrobacterium were then collected and re-suspended in infection liquid medium (containing MS basal salts and B5 vitamins, sucrose 20 g/L, glucose 10 g/L, MES, 4 g/L, zeatin riboside 2 mg/L, 3-10 mg/L TDZ, 80 mg/L acetosyringone (AS), 150 mg/L dithiothreitol (DTT), pH=5.5). The concentration of the Agrobacterium was adjusted until OD660=0.6.

### (3) Preparation and Infection of Explants

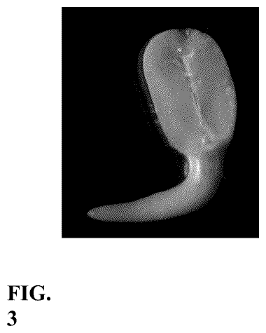

One cotyledon of the split germinated seeds was removed with a scalpel to expose the shoot apex with its two primary leaves. The two primary leaves were completely removed to expose the apical meristem and the apical region was gently wounded with the sharp end of a scalpel. See FIG. 3. The resultant explants were then placed in an Agrobacterium suspension and add 1/1000 10% F68; the suspension was then placed under vacuum at 800-900-mbar for 5-30 min followed by sonication at 45 kHz for 30-60s with gentle shaking. The explants were inoculated for 2-3 hours with the Agrobacterium with 50-80 rpm shaking.

### (4) Co-Culture

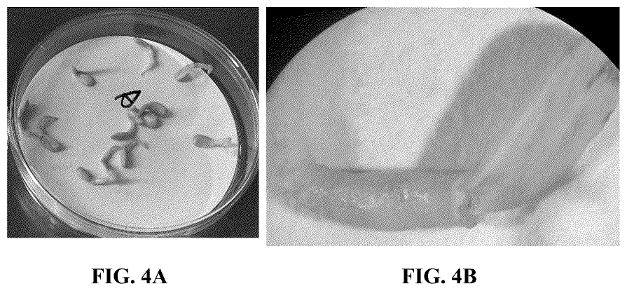

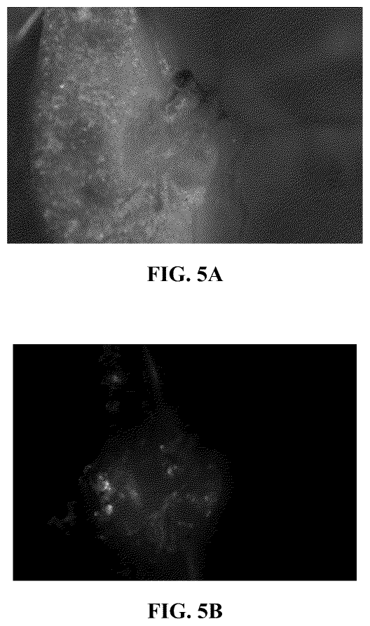

After inoculation in (3), the Agrobacterium suspension was removed, and the explants were then placed onto a piece of filter paper in a petri dish which prewetted by 0.5 ml co-cultivation media (MS basal salts and vitamins, MS iron, zeatin 1 mg/L, sucrose 10 g/L, glucose 5 g/L, MES 2 g/L, acetosyringone 20 mg/L, silver nitrate 0.5 mg/L) with the adaxial side up. One side of the filter paper was covered, and the petri dish was sealed with parafilm. Approximately 10~20 explants were placed per petri dish. The explant and Agrobacterium were co-cultured for 4 days in low-illumination condition: PAR (photosynthetically active radiation) 40 μmol m−2 s−1 with 16/8 light/dark condition. The plate containing explants was covered with a steel tray in an incubator (PERVICAL® Scientific Chambers, Perry, IA). These conditions were chosen to increase infection and prevent embryo deterioration. The explants were then co-cultured at 22° C. in the dark for 3-6 days in growth chamber. See FIGS. 4-5.

### (5) Regeneration

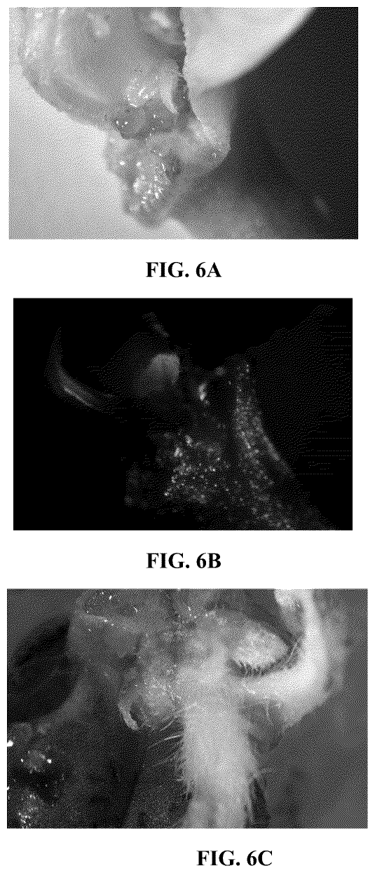

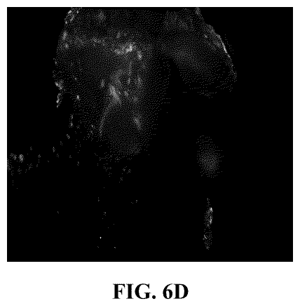

After co-culture, the explants were transferred onto SFR1 medium for shoot regeneration (Gamborg B5 basal salts and B5 vitamins, MS Iron 200×, sucrose 20 g/L, MES 1 g/L, timentin 150 mg/L, carbenicillin 150 mg/L, agar 7.5 g/L, lipoic acid-50 mM, BAP 1 mg/L). The SFR1 medium also contained a selection agent (i.e., spectinomycin 50-150 mg/L). The explants were allowed to culture on SFR1 media in the dark at 25° C. for 3-6 days, and then grown under light (16/8 light/dark) at 25° C. for 9-14 days. FIGS. 6A-D show formation of the primary shoots with GFP expression.

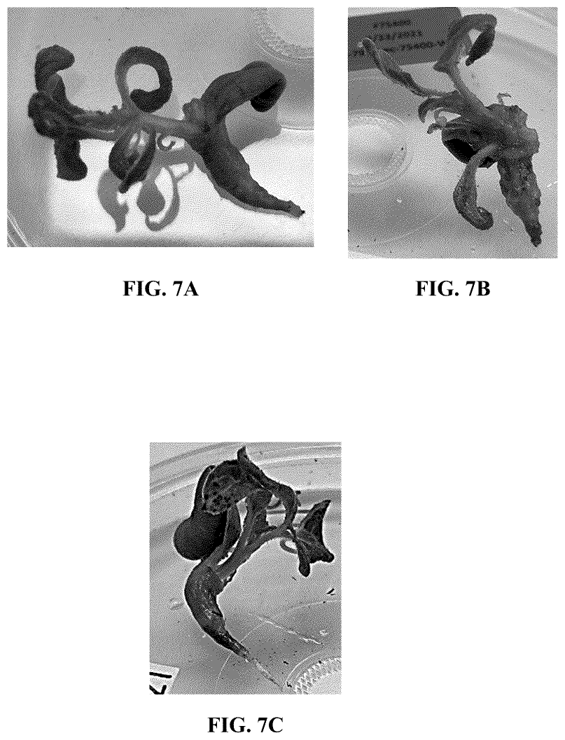

After selection on SFR1 media with the selection agent, explants with shoots were transferred onto the selection-free SFR1 media and grown for 7-14 days. When the explants produced shoots with complete apical meristem, they were ready for transplanting to soil. See FIGS. 7A and 7B.

(6) Transplant of Explants into Soil

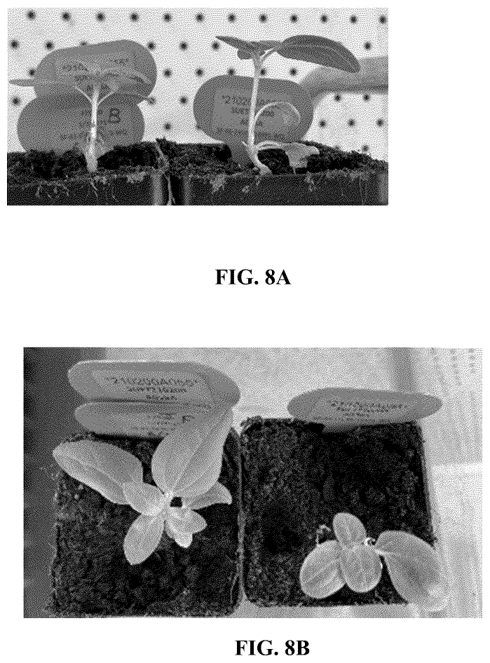

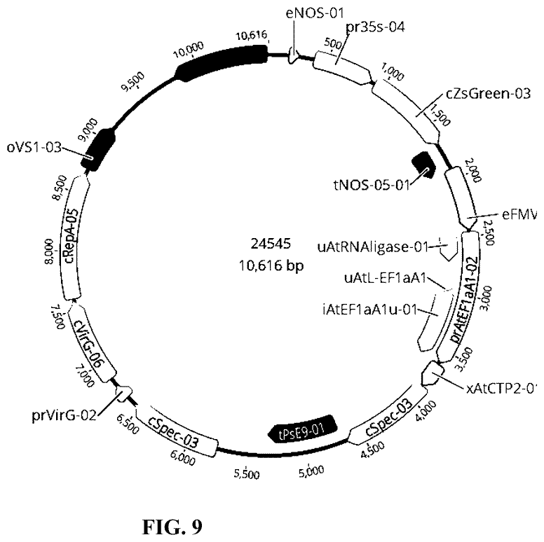

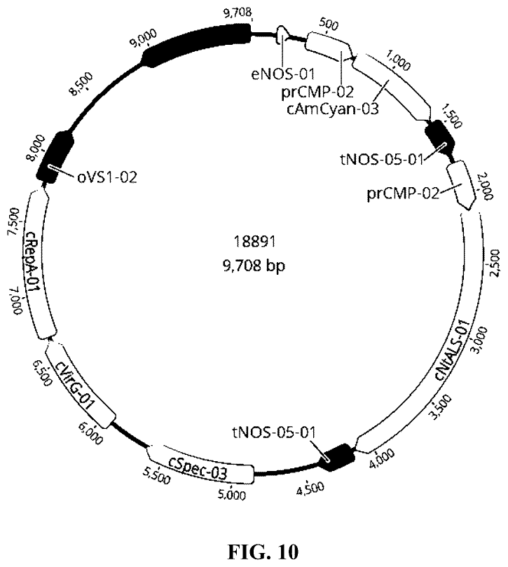

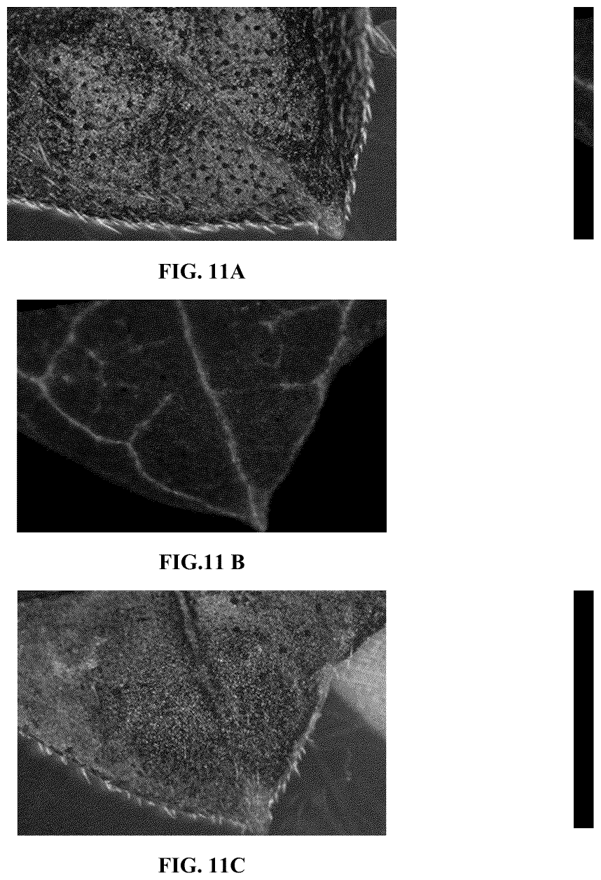

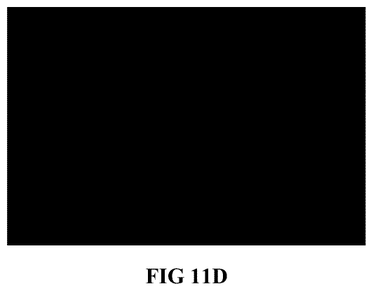

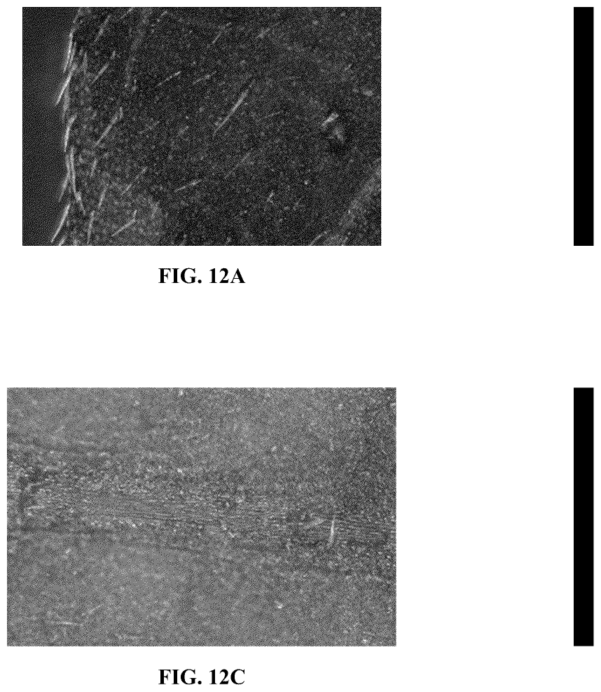

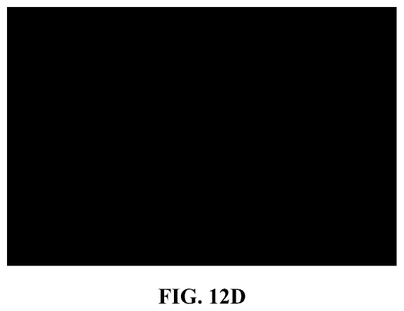

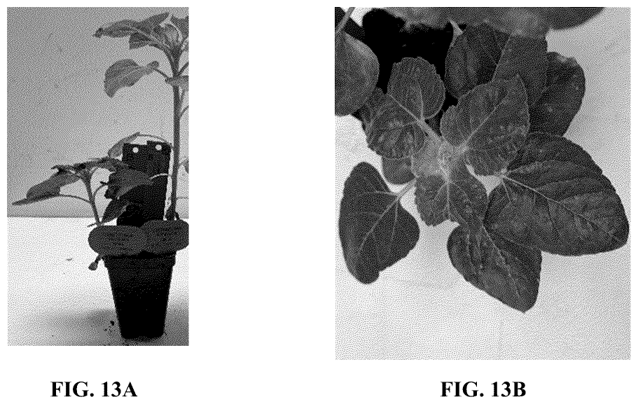

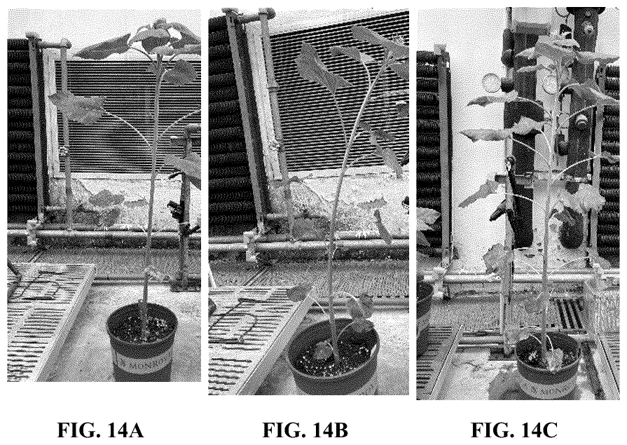

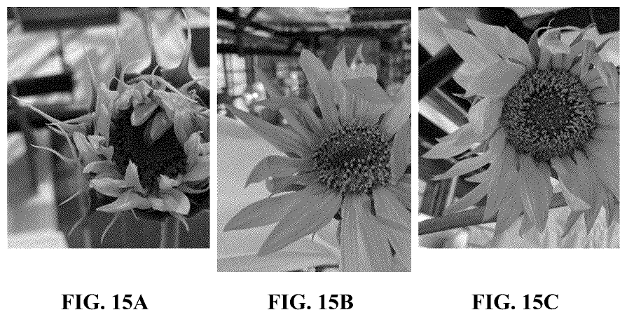

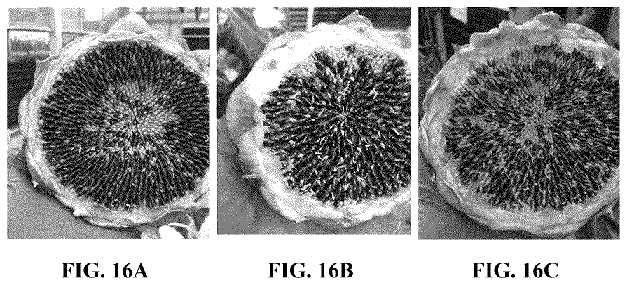

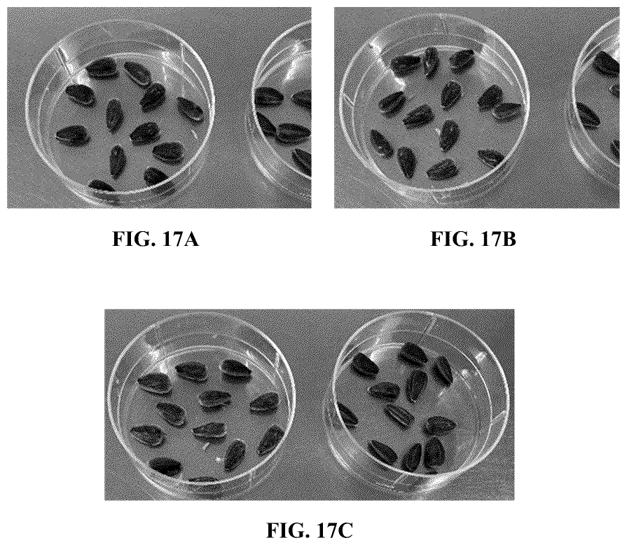

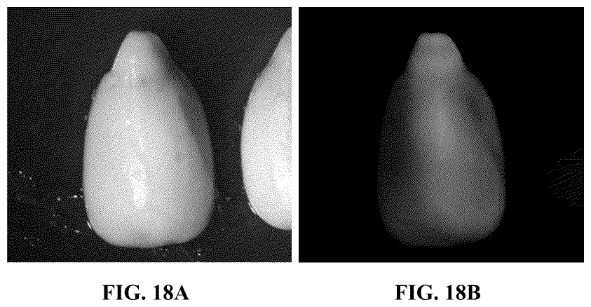

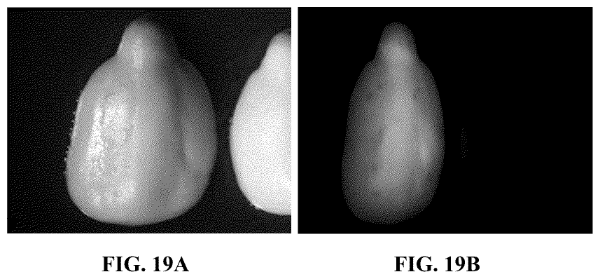

Explants with shoot meristem were selected for transplantation into germination trays in GH chamber rooms (soil mixture as substrate). Each tray was covered with a plastic domed lid and put on seedling shelf with the following growth condition: 27° C. in daytime for 20 hours and 20° C. at night for 4 hours. See FIGS. 7-8. For ALS transgenic plants and growth in a greenhouse for 3-4 months, T1 seeds from transgenic plants were obtained. FIG. 16. Most transformed sunflower plants had over two hundred seeds, and the seed size and full seeds rate were close to seeds of wild type plants. See FIG. 16; Table 4. GFP or CFP expression was detected with a fluorescence stereo microscope. See FIGS. 18 and 19.

TABLE 4
Seeds from T0 events

| | | | | | Seeds |
|---|---|---|---|---|---|
| | | Selection | # of | Flower | number |
| Plant ID | Vector | system | Seeds | diameter | (kernel) |
| SUET210201A008A | 18891 | ALS | 220 | 20 CM | 401 |
| SUET210200A002A | 24545 | Spec | 180 | 15 CM | 212 |
| SUET210200A003A | 24545 | Spec | 41 | 15 CM | 38 |
| SUET210200A004A | 24545 | Spec | 180 | 15 CM | 204 |
| SUET210200A005A | 24545 | Spec | 200 | 20 CM | 265 |

### 7. Transgene Inheritance

Some T1 seeds of transgenic plants were sowed, then a TaqMan assay was used to detect the transgene inheritance. Positive plants with T-DNA from all the plants germinated from Ti seeds were observed. Accordingly, all TO events tested were able to inherit transgene to next generation. The results of this testing are summarized in Table 5.

TABLE 5
Transgene inheritance confirmed by TaqMan

| | | | # of | # of | Positive |
|---|---|---|---|---|---|
| | | Selection | E1 | Positive | rate |
| Plant ID | Vector | system | Plants | Events | (%) |
| SUET210201A008A | 18891 | ALS | 6 | 3 | 50% |
| SUET210200A002A | 24545 | Spec | 8 | 8 | 100% |
| SUET210200A003A | 24545 | Spec | 4 | 4 | 100% |
| SUET210200A004A | 24545 | Spec | 5 | 5 | 100% |
| SUET210200A005A | 24545 | Spec | 6 | 5 | 83% |

### 8. Reproducible of Transformation Protocol

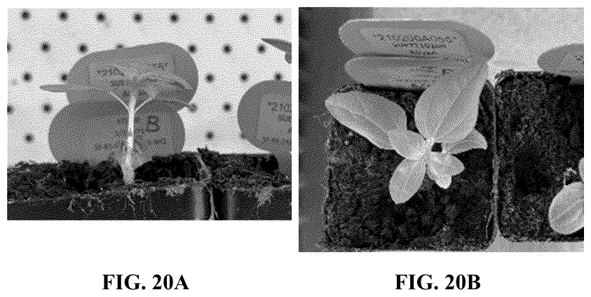

The protocol described above was repeated and transgenic plants (events) were repeatedly obtained. These transgenic plants (events) were observed to grow in a green house without abnormal phenotyping. See FIG. 20. As is evident from the data in Table 6, the transformation system was observed to be reproducible.

TABLE 6
Results from Additional Transformation Experiments

| Experiment No. | # of Explants | # of Positive Events | TF |
|---|---|---|---|
| SF-78-SP-24545-C | 37 | 1 | 2.7% |
| SF-79-SP-24545-A | 40 | 1 | 2.5% |
| SF-81-SP-24545-B | 42 | 3 | 7.1% |

### 9. Impact of Plant Growth Regulator TDZ

Different concentrations of TDZ in the infection medium were tested to assess if TDZ concentration impacts transformation (Experiment alias: SF-52-SP-24545, SF-66-SP-24545, SF-81-SP-24545; variety: F75400). The process of transformation with explants from 1-day imbibed seeds was as described above. TDZ was added in the infection medium during the infection process for 2-4 hours. A TDZ concentration of 3-10 mg/L was used. TDZ was observed to significantly improve shoot induction rate and transformation efficiency. The results of this testing are summarized in Table 7 below.

TABLE 7
Transformation efficiency with different concentration of TDZ

| | TDZ in infection | # of | # of TaqMan | |
|---|---|---|---|---|
| Alias | medium | Explants | Positive Events | TF |
| SF-52-SP-24545-C | 0 mg/L | 74 | 0 | 0 |
| SF-52-SP-24545-D | 3 mg/L | 65 | 1 | 1.5% |
| SF-66-SP-24545-B | 4 mg/L | 49 | 4 | 8.2% |
| SF-81-SP-24545-B | 8 mg/L | 42 | 3 | 7.1% |
| SF-78-SP-24545-C | 10 mg/L | 37 | 1 | 2.7% |

### 10. Impact of Durations of Selection

The number of selection days was varied to determine if there is any impact on transformation (Experiment reference: SF-66-SP-24545, SF-52-SP-24545, SF-54-SP-24545; variety: F75400; vector: 24545).

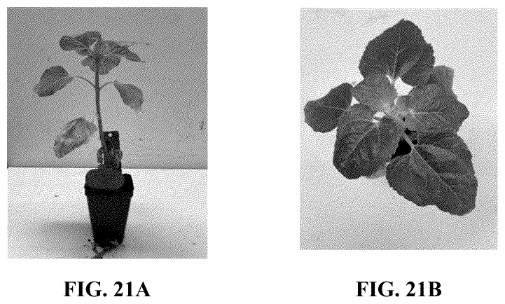

For this testing, the process of transformation with explants from 1-day imbibed seeds was as described above. The results of this testing are shown in Table 8 and FIG. 21.

Regeneration on SFR1 medium with selection agent after co-culture was observed to be important. Selection for 4 days, resulted in no transformants. In this experiment, the best timing in selection was observed to be 9 days, which resulted in a TF of 8.2%. Table 8, FIG. 21. Accordingly, the duration of selection with selection agent on medium is not shorter than 4 days.

TABLE 8
Transformation with different selection days

| | | # of | # of TaqMan | | Selection |
|---|---|---|---|---|---|
| | # of | Regenerate | Positive | | on |
| Alias | Explants | shoots | events | TF | medium |
| SF-64-SP-24545 | 102 | 0 | 0 | 0 | 4 days |
| SF-66-SP-24545-B | 49 | 6 | 4 | 8.2% | 9 days |

The Examples illustrate embodiments of the disclosed methods for genetic transformation of sunflower, which is based on Agrobacterium-mediated gene delivery to meristem cells of freshly imbibed/germinated sunflower seeds.

As the Examples indicate, multiple transgenic-shoot structures were induced from infected explant tissues. Then the explants were shortly selected on selection media with selection agent (7-14 days, Spectinomycin as selection agent) and transplanted into soil to allow shoot and root development under controlled conditions (in growth chamber). From multiple experiments, transgenic events were successfully generated with aadA or ALS as selectable markers (spectinomycin and bensulfuron-methyl as selection agents respectively). The transformation frequency (TF) is 8.2% for spectinomycin selection, and 3.6% for bensulfuron-methyl selection system.

The TO transgenic events generated from this method are normal and healthy without abnormal phenotype or developmental issues (e.g., early flowering, rootless), the TO events can produce >200 seeds per plant. The timeline of this sunflower transformation method takes only about 8 weeks (with 4-week in vitro process and 4-weeks GH steps), which is much shorter than conventional transformation methods usually taking 3 months. Accordingly, this disclosure provides rapid sunflower stable transformation for genetic manipulation or genome editing.

### Example 3: Additional Sunflower Transformation Protocol (Protocol V2)

Mature sunflower seeds were transformed using the protocol shown in FIG. 2A. The protocol for transformation is descried in detail below. The protocol does not require a germination process (overnight imbibe seeds), rather it relies on direct transformation of mature seeds. The protocol has six general steps which are described below.

### (1) Seed Germination

Seeds were allowed to imbibe for 4-48 hour, ideally about 20 hours prior isolation of the explant.

### (2) Agrobacterium Preparation

Agrobacterium from storage at −80° C. were streaked to YP (Yeast Peptone) medium with 100 mg/L spectinomycin and 50 mg/L kanamycin at least 3 days before infection. They were re-streaked to a new YP medium plate with some concentrations of spectinomycin and kanamycin 1 day before infection. The Agrobacterium were then collected and re-suspended in infection liquid medium (containing MS basal salts and B5 vitamins, sucrose 20 g/L, glucose 10 g/L, MES, 4 g/L, zeatin riboside 2 mg/L, 3-10 mg/L TDZ, 80 mg/L acetosyringone (AS), 150 mg/L dithiothreitol (DTT), pH=5.5). The concentration of the Agrobacterium was adjusted until OD660=0.6.

### (3) Preparation and Infection of Explants

To isolate the explant, the imbibed seed was split, one cotyledon was removed, primary leaves were removed, a wound across the apical region was made, and the whole hypocotyl was kept thereby generating the explant. The method does not require use of induced/existed axillary buds/apical meristem before inoculation. The primary meristem was physically destroyed before inoculation (infection). The resultant explant was infected with Agrobacterium by culturing the explant in the presence of 3-20 mg/L thidiazuron (TDZ) in infection medium. Specifically, to minimize the negative effects of long existing of TDZ during tissue culture., the higher concentration of TDZ (3-20 mg/L) in our Agrobacterium suspension was used only during inoculation (<4 hours) and removed afterwards to induce shoots during co-culture and later tissue culture steps. The infection involved using sonication at 45 KHz for one minute and vacuum for thirty minutes.

### (4) Co-Culture

After inoculation, the Agrobacterium suspension was removed, and the explants were then placed onto a piece of filter paper in a petri dish which prewetted by 0.5 ml co-cultivation media (MS basal salts and vitamins, MS iron, zeatin 1 mg/L, sucrose 10 g/L, glucose 5 g/L, MES 2 g/L, acetosyringone 20 mg/L, silver nitrate 0.5 mg/L) with the adaxial side up. One side of the filter paper was covered, and the petri dish was sealed with parafilm. Approximately 10~20 explants were placed per petri dish. The explant and Agrobacterium were co-cultured for 4 days in low-illumination condition: PAR (photosynthetically active radiation) 40 μmol m⁻² s⁻¹ with 16/8 light/dark condition. We covered the plate containing explants with steel tray in an incubator (PERVICAL® Scientific Chambers, Perry, IA) which is set 22° C., the light module is SciWhite™ LED tiles.

### (5) Regeneration

Transformed sunflower explants was transferred onto regeneration media by inserting cotyledon with meristem region into solid medium with a selection agent. Hypocotyl was left untouching the medium. The hypocotyl was covered with sterilized matrix and put the jar in 22° C., 16/8 h light/dark. The regeneration step lasted for 7-14 days. Specifically, after co-culture, the mature seeds were placed on regeneration medium as follows: cotyledon/meristem region was inserted into solid medium with a selection agent (100 mg/L spectinomycin), the hypocotyl was left upside (the whole seeds were upside down); the container was covered with sterilized matrix (soil mixture) and placed in growth chamber for inducing shooting and rooting simultaneously under short time selection (7-14 days).

### (6) Rooting

Subsequentially, the transformed explants were moved to rooting media for root induction and development. Specifically, the explants were picked out of regeneration medium, and then put into rooting medium lacking a selection agent. The explants were grown in the rooting medium for 3-14 days until shoots and roots generated.

(7) Transplant of Explants into Soil

After the shoots and roots were generated, the plants were transplanted into soil and grown for 2-4 weeks covered by a plastic dorm lid for keep the humidity and lighting. Only plants with good shoots and roots were transplanted into soil and grown in greenhouse without any selection. The plants were subsequently characterized.

1. Rooting Rate Improved from Method Version 1 (Described in Example 2) to Version 2 (Described in Example 3)

Generally rooted plantlet is easier to survive than the one without roots in greenhouse. The rooting rate increased from <10% to >75% in average which significantly increased survival rate after plants transplanting to soil. The protocol is applicable to another germplasm, for example it is demonstrated in variety FS5698B, 88% rooting frequencies were achieved.

TABLE 9
Rooting rate of method version 1 and version 2

| Experiment | | | # of | # of Rooted | Rooting |
|---|---|---|---|---|---|
| No. | Variety | Method | Explants | explants | rate |
| 1 | F75400 | V2 | 111 | 106 | 95% |
| 2 | F75400 | V2 | 33 | 25 | 76% |
| 3 | F75400 | V2 | 25 | 16 | 64% |
| 4 | FS5698B | V2 | 33 | 29 | 88% |
| 5 | F75400 | V1 | 45 | <5 | <10% |

Transformation Frequency Improvement of the Protocol V2 from the Protocol V1

Table 10 is the results of transformation experiment using F75400, the transformation frequencies were side by side compared for transformation protocol version 1 and version 2, significant TF improvements was observed.

TABLE 10
TF of method version 1 and version 2

| Transformation | # of | # of Positive | | Low | Backbone |
|---|---|---|---|---|---|
| method | Explants | events | TF | copy | free |
| V2 | 48 | 7 | 15% | 85.7% | 57.1% |
| V1 | 45 | 1 | 2.7% | 1 | 1 |

### 3. Reproducibility of Rapid Transformation Protocol of Improved Method

To test the reproducibility of improved transformation protocol V2, the variety F75400 and construct 24545 were used. The positive events were regenerated with normal shoots and roots, and further confirmed by molecular analysis. Table 11 below shows the reproducibility of transformation protocol and duration of the whole process which ranged from 41 days to 56 days; demonstrating how rapid and reliable the new protocol is.

TABLE 11
TF of method version 2 with repeatable experiments

| Experiment | # of | # of Positive | | |
|---|---|---|---|---|
| No. | Explants | events | TF | Timeline/days |
| SF02 B | 52 | 3 | 6% | 41 |
| SF03 C | 45 | 11 | 24% | 54 |
| SF95 A | 48 | 6 | 13% | 48 |
| SF08 A | 48 | 6 | 13% | 55 |
| SF09 A | 45 | 7 | 16% | 54 |

### 4. Cytokinin Preconditioning (e.g., TDZ but not Limited to TDZ)

Adding different cytokinin into germination medium can improve shoot induction rate and TF. The resistant plantlets were generated with normal shoots and roots, which was further confirmed by visualizing green fluorescent protein expression (GFP). (Experiment alias: SF-52-SP-24545, SF-66-SP-24545, SF-81-SP-24545; variety: F75400).

TABLE 12
Transformation efficiency with different cytokinin in germination

| | | | | | # of | Rate of |
|---|---|---|---|---|---|---|
| | | | | | Resistant | resistant |
| | | | No. | Resistant | plants | plants |
| | Hormone | # of | resistant | shoot | with | with |
| TID | in Germ | Explants | shoot | rate | GFP | GFP (%) |
| A | control | 76 | 2 | 3% | 1 | 2% |
| | (0 mg/L) | | | | | |
| B | TDZ | 68 | 7 | 10% | 5 | 7% |
| | 4 mg/L | | | | | |
| C | BAP | 71 | 6 | 8% | 5 | 7% |
| | 5 mg/L | | | | | |

### 5. Duration of Seeds Germination:

Seeds imbibed in medium for 4-48 hours can increase spectinomycin resistant shoot induction frequencies. (Experiment alias: SF-55-SP-24545, variety: F70219; SF-23-SP-24545, SF-22-SP-24545, variety: F75400).

TABLE 13
Transformation efficiency with different germination time

| | | Imbedded | | No. | Resistant |
|---|---|---|---|---|---|
| | | time/ | # of | resistant | shoots |
| TID | Variety | hours | Explants | shoots | rate |
| SF-55-SP-24545-A | F70219 | 20 | 64 | 8 | 13% |
| SF-55-SP-24545-C | F70219 | 4 | 45 | 10 | 22% |
| SF-23-SP-24545-A | F75400 | 20 | 70 | 7 | 9% |
| SF-22-SP-24545-A | F75400 | 48 | 89 | 10 | 11% |

6. Range of Spectinomycin Concentration in Selection Medium (Spec 100 mg/L-800 mg/L);

Selection medium with spectinomycin 100-800 mg/L was tested. The resistant plantlets are generated with normal shoots and roots, further confirmed by visualizing GFP. All treatments of spectinomycin concentrations ranged from 100 mg/L to 800 mg/L generated resistant shoots. (Experiment alias: SF-23-SP-24545; variety: F75400).

TABLE 14
Resistant frequencies of different concentration of spectinomycin

| | | | | | # of | Rate of |
|---|---|---|---|---|---|---|
| | | | | | Resistant | resistant |
| Treatment | Spectinomycin | # of | Shooting | Rooting | plants with | plants with GFP |
| ID | mg/L | Explants | Rate (%) | rate (%) | GFP | (%) |
| SF23A | 100 | 89 | 40% | 66% | 10 | 11% |
| SF23B | 200 | 67 | 18% | 36% | 6 | 9% |
| SF23C | 300 | 61 | 16% | 30% | 2 | 3% |
| SF23D | 500 | 68 | 10% | 18% | 2 | 3% |
| SF23E | 800 | 70 | 9% | 9% | 1 | 1% |

### 7. Genotype Independent

The flexibility of transformation protocol was evaluated in multiple sunflower elite lines with V2 protocol. The positive events are generated with normal shoots and roots, confirmed by visualizing GFP. The data shows nine more elite lines are transformable with method V2 besides the model line F75400 and another line F70219.

TABLE 15
Transformation results in multiple sunflower elite lines

| | | | | Rate of |
|---|---|---|---|---|
| | | | # of | resistant |
| | | | Resistant | plants |
| Experiment | | # of | plants | with |
| No. | Variety | Explants | with GFP | GFP (%) |
| SF13 A | F715 | 15 | 8 | / |
| SF13 C | B531HOIMI-2 | 72 | 4 | 6% |
| SF13 D | BD40713MH | 95 | 2 | 2% |
| SF14 D | F73100 | 95 | 9 | 9% |
| SF15 A | FR84205 | 105 | 1 | 1% |
| SF15 D | RD506011MH | 96 | 9 | 9% |
| SF16 A | RW666RM1 | 114 | 8 | 7% |
| SF16 C | F75800 | 84 | 2 | 2% |
| SF16 E | FS5698B | 84 | 2 | 2% |

It is to be understood that while the disclosure has been described in conjunction with the preferred specific embodiments thereof, that the foregoing description and the examples that follow are intended to illustrate and not limit the scope of the disclosure. It will be understood by those skilled in the art that various changes may be made, and equivalents may be substituted without departing from the scope of the disclosure; and that other aspects, advantages, and modifications will be apparent to those skilled in the art to which the disclosure pertains. In addition to the embodiments described herein, the present disclosure contemplates and claims those inventions resulting from the combination of features of the disclosure cited herein and those of the cited prior art references which complement the features of the present disclosure. Similarly, it will be appreciated that any described material, feature, or article may be used in combination with any other material, feature, or article, and such combinations are considered within the scope of this disclosure.

1. A method of directly transforming a Helianthus annuus plant with a heterologous polynucleotide from a mature seed comprising:
a) generating an explant from a germinated a Helianthus annuus plant seed by isolating a cotyledon and primary leaves with complete meristem and shoot radical;
b) contacting the Helianthus annuus explant comprising the cotyledon with an infection bacterium comprising a heterologous polynucleotide in an infection medium supplemented with a plant hormone;
c) co-culturing the explant from a germinated a Helianthus annuus plant in the presence of the infection bacterium in a medium lacking the plant hormone;
d) culturing the mature seed explant in a regeneration medium comprising a selection agent;
e) culturing the seed with regenerated shoot with secondary roots in a medium lacking a selection agent; and
f) transplanting the explants into soil under conditions allowing for further development.

2. The method of claim 1, further comprising germinating a Helianthus annuus plant seed for about a day.

3. The method of claim 1, wherein the infection bacterium comprises Agrobacterium tumefaciens.

4. The method of claim 1, wherein the infection bacterium is modified to comprise the heterologous polynucleotide.

5. The method of claim 4, wherein the infection bacterium is modified by transformation.

6. The method of claim 1, wherein the heterologous polynucleotide is a gene.

7. The method of claim 1, wherein the Helianthus annuus explant is contacted with the infection bacterium using a vacuum and sonication.

8. The method of claim 7, wherein the vacuum and sonication enhance infection efficiency.

9. The method of claim 1, wherein the plant hormone comprises one or more of TDZ, BAP, zeatin, and kinetin.

10. The method of claim 9, wherein the plant hormone comprises a concentration of about 3-20 mg/ml.

11. The method of claim 1, comprising contacting the Helianthus annuus with an infection medium containing the plant hormone for about 4 hours or less.

12. The method of claim 1, wherein the co-culturing comprises culturing with or without light.

13. The method of claim 12, wherein the co-culturing comprises culturing under low light.

14. The method of claim 12 or 13, comprising co-culturing for about 4-6 days.

15. The method of claim 1, wherein the culturing the mature seed explant in a regeneration medium comprises inserting the cotyledon/meristem region into a solid medium in the presence of a selection agent, covering the seed with matrix and growing the seeds.

16. The method of claim 15, comprising culturing for a duration of about 7-14 days.

17. The method of claim 16, comprising culturing in dark for about the first 2-3 days.

18. The method of claim 15, wherein every 24 hours of culturing comprises culturing for 16 hours in the presence of light and 8 hours in the presence of dark.

19. The method of claim 1, wherein the culturing the seed with regenerated shoot with secondary roots in a medium lacking a selection agent comprises culturing for a duration of about 3-7 days.

20. The method of claim 1, wherein the transplanting the explants into soil under conditions allowing for further development comprises growing the explants for about 2-3 weeks without a selection agent.

21. The method of claim 1, wherein the transplanting the explants into soil under conditions allowing for further development comprises growing the explants with a selection agent.

22. The method of any one of claims 1-20, further comprising characterizing the transgenic plant.

23. The method of claim 21, further comprising determining the presence of the heterologous polynucleotide in the transgenic plant.

24. The method of claim 1, wherein the selection agent comprises an antibiotic, wherein the selection agent comprises spectinomycin, bensulfuron-methyl, or one or more combinations thereof.

25. The method of claim 24, wherein the selection agent comprises spectinomycin, bensulfuron-methyl, or one or more combinations thereof.

26. A plant or plant part produced by the method of claim 1.

27. A progeny seed produced by: a) crossing the plant of claim 26 with a second plant or b) selfing the plant of claim 26.

28. A derivative or commodity product produced or obtained from the plant or plant part of claim 26
